(19) United States
(12) Patent Application Publication
(10) Pub. No.: US 2009/0215710 A1
(43) Pub. Date: Aug. 27, 2009

(54) CARBOHYDRATE BASED TOLL-LIKE RECEPTOR (TLR) ANTAGONISTS

(75) Inventors: Shakti Upadhyay, Navi Mumbai (IN); Ashwani Sharma, Navi Mumbai (IN); Anuradha Sharma, Navi Mumbai (IN); Vikram Rajagopal, Navi Mumbai (IN); Praneel Datla, Navi Mumbai (IN); Akshaya Bellary, Navi Mumbai (IN); Shiva Prasad Singh, Navi Mumbai (IN)

Correspondence Address: MINTZ, LEVIN, COHN, FERRIS, GLOVSKY AND POPEO, P.C, 5 Palo Alto Square - 6th Floor, 3000 El Camino Real, PALO ALTO, CA, 94306-2155, US

(73) Assignee: RELIANCE LIFE SCIENCES PVT. LTD., Navi Mumbai (IN)

(21) Appl. No.: 12/236,358

(22) Filed: Sep. 23, 2008

(30) Foreign Application Priority Data
Sep. 24, 2007 (IN) 1854/MUM/2007
Dec. 24, 2007 (IN) 2542/MUM/2007

(51) Int. Cl. A61K 31/7028; C07H 5/06; C12N 5/06; C12Q 1/02

(52) U.S. Cl. 514/42; 536/29.1; 435/375; 435/29

(57) ABSTRACT

The invention provides carbohydrate based compounds, methods of preparation, and compositions useful for modulating signaling through Toll-like receptors. The methods involve contacting a TLR-expressing cell with a carbohydrate based compound of the invention having a core structure comprising of one or more sugar moieties. The carbohydrate based compounds are useful for inhibiting immune stimulation involving TLR ligands, especially TLR4 and TLR2. The compounds also are suitable for inhibition of inflammatory conditions resulting from infections. The compounds have use in the treatment of inflammation, autoimmunity, allergy, asthma, graft rejection, graft versus host disease, infection, sepsis, cancer, and immunodeficiency.

### CROSS-REFERENCE TO RELATED PATENT APPLICATIONS

The present application claims benefit of Indian Provisional Patent Application No. 1854/MUM/2007 filed Sep. 24, 2007, and Indian Provisional Patent Application No. 2542/MUM/2007 filed on Dec. 24, 2007 which are incorporated herein by reference in their entirety.

### FIELD OF THE INVENTION

The present invention relates to the synthesis of novel carbohydrate based molecules, their in-vitro studies and modulation of immune functions mediated through Toll-like receptor (TLR) molecules.

### BACKGROUND OF THE INVENTION

The innate or natural immune system recognizes a wide spectrum of pathogens without a need for prior exposure. The main cells responsible for innate immunity are monocytes/macrophages and neutrophils which phagocytose microbial pathogens that trigger the innate, inflammatory, and specific immune responses.

Humans have evolutionarily conserved immune receptors, Toll-like receptors (TLRs), which line up as the first defenses against the invading foreign pathogens. The TLRs belong to a family of pattern recognition receptors (PRR) involved in the recognition of a wide range of microbial molecules shared only by pathogens and significantly distinguishable from host molecules. These are collectively referred to as pathogen-associated molecular patterns (PAMP) and comprise compounds such as Lipopolysaccharides (LPS) from Gram-negative bacteria and peptidoglycan from Gram-positive bacteria.

The prototype receptor Toll was first identified in the fruit fly Drosophila but later found in mammals, particularly on mononuclear phagocytes. Currently there are thirteen TLRs (named simply TLR1 to TLR13) identified both in human and mice with many equivalent forms subsequently identified in other related mammalian species. Of the TLRs identified, for most of them, except TLR10, the natural ligands have been identified. These include various proteins, lipopeptides (LP), lipoteichoic acid, lipopolysaccharides (LPS), and oligonucleotides (double-stranded RNA, single-stranded RNA, and DNA, Kawai T, Akira S. TLR signaling. Cell Death Differ. 2006; 13: 816-25.). These receptors are a class of single membrane-spanning non-catalytic receptors forming a receptor superfamily with the Interleukin-1 receptors (Interleukin-1 Receptor/Toll-Like Receptor Superfamily) due to a common so called TIR (Toll-IL-1 receptor) domain. TLRs exert their functioning by forming dimers which might be homodimers heterodimers as observed in case of TLR2 with TLR1 or TLR6, each dimer having a different ligand specificity.

The discovery of the Toll-like receptors finally identified the innate immune receptors that were responsible for many of the innate immune functions that had been studied for many years. Interestingly, TLRs seem only to be involved in the cytokine production and cellular activation in response to microbes, and do not play a significant role in the adhesion and phagocytosis of microorganisms. Binding of TLR leads to the production of inflammatory cytokines, including Tumour Necrosis Factor-alpha (TNF-α), capable of exerting host-damaging effects as seen in conditions like sepsis and fever syndromes, as well as in autoimmune diseases such as rheumatic arthritis and inflammatory bowel disease (IBD). (Raza A. Anti-TNF therapies in rheumatoid arthritis, Crohn's disease, sepsis, and myelodysplastic syndromes. Microsc Res Tech. 2000; 50: 229-35; Hehlgans T, Pfeffer K. The intriguing biology of the tumour necrosis factor/tumour necrosis factor receptor superfamily: players, rules and the games. Immunology. 2005. 115: 1-20.). TLR signaling also releases IL-12 and enhances the cells' antimicrobial killing mechanisms and antigen presenting capacity. The function of the TLRs was discovered by Beutler and colleagues, (Poltorak A, He X, Smirnova I, Liu M Y, Van Huffel C, Du X, Birdwell D, Alejos E, Silva M, Galanos C, Freudenberg M, Ricciardi-Castagnoli P, Layton B, Beutler B. Defective LPS signaling in C3H/HeJ and C57BL/10ScCr mice: mutations in Tlr4 gene. Science. 1998; 282: 2085-8.). These workers used positional cloning to prove that mice that could not respond to LPS had mutations that abolished the function of TLR4. This identified TLR4 as a key component of the receptor for LPS, and strongly suggested that other Toll-like receptors might detect other signature molecules of microbes, such as those mentioned above.

The significance of the toll like receptors in the immune response to LPS has further been demonstrated specifically in two receptors TLR2 and TLR4. Yang et. al and Kirschning et. al have clearly demonstrated that Toll-like receptor 2 (TLR2) is a signaling receptor activated by LPS and depending on a LPS-binding protein and is enhanced by CD14. (Yang R B, Mark M R, Gray A, Huang A, Xie M H, Zhang M, Goddard A, Wood W I, Gurney A L, Godowski P J. Toll-like receptor-2 mediates lipopolysaccharide-induced cellular signalling. Nature. 1998; 395: 284-8; Kirschning C J, Wesche H, Merrill Ayres T, Rothe M. Human toll-like receptor 2 confers responsiveness to bacterial lipopolysaccharide. J Exp Med. 1998; 188: 2091-7.) Further, they showed that following LPS stimulation of TLR2, an interleukin 1 receptor-like NF-kappaB signaling cascade was initiated. Further, reports by Poltorak et. al and Quereshi et al concluded that TLR4 was required for a response to LPS and mutations in them would result in defective LPS signaling or endotoxin tolerance. (Poltorak A, et al., Science. 1998; 282: 2085-2088; Qureshi S T, Larivière L, Leveque G, Clermont S, Moore K J, Gros P, Malo D. Endotoxin-tolerant mice have mutations in Toll-like receptor 4 (Tlr4) J Exp Med. 1999; 189: 615-625).

Although LPS is an immunomodulatory agent, its medicinal use is limited due to its extreme toxicity including the induction of systemic inflammatory response syndrome. The biologically active endotoxio sub-structural moeity of LPS is lipid-A, a phosphorylated, multiple fatty acid acylated glucosamine disaccharide that serves to anchor the entire structure in the outer membrane of the gram-negative bacteria. The toxic effects of the lipid A was addressed by selective chemical modification of the lipid A to produce monophosphoryl lipid A compounds (MPL®: vaccine adjuvant and immunostimulant from Corixa (Seattle, Wash., US) and structurally like MPL® compounds) which is described in U.S. Pat. Nos. 4,436,727; 4,877,611; 4,866,034; 4,912,094; 4,987,237; and Johnson et al. (Johnson D A, Keegan D S, Sowell C G, Livesay M T, Johnson C L, Taubner L M, Harris A, Myers K R, Thompson J D, Gustafson G L, Rhodes M J, Ulrich J T, Ward J R, Yorgensen Y M, Cantrell J L, Brookshire V G. 3-O-Desacyl monophosphoryl lipid A derivatives: synthesis and immunostimulant activities. J Med Chem. 1999; 42: 4640-49).

Considering the basis of the MPL® immunostimulant and other bacterial cell wall components, a family of novel synthetic compounds, the aminoalkyl glucosaminide phosphates (AGPs) were developed. AGP compounds were found to be adjuvants and immunoeffectors, increasing the efficiency in existing vaccines. These AGP compounds belong to the class of synthetic mono and disaccharide mimetics of monophosphoryl lipid A as described in U.S. Pat. No. 6,303,347 and in WO 98/50399. These compounds, besides being synthetic in nature, have improved toxicity profiles as compared to monophosphoryl lipid A. These compounds have been used in combination with antigens in vaccine formulations (U.S. Pat. No. 6,113,918) and also as monotherapies in absence of antigens (WO 01/90129).

Based on the above various aminoalkyl glucosaminide phosphate compounds have been described in U.S. Pub. Patent App. 2005/0227943. Cyclic AGPs have been described in PCT Patent PCT/US01/24284 and azacycloalkyl AGPs have been described in U.S. Pat. Nos. 6,911,434 and 6,800,613. Further, specific glycosaminoglycan polymers (GAG molecules) have been disclosed in U.S. Pub. Patent App. 2005/0272696 which have differential effects on cancer and hence have been designed for “personalised medicine”.

Further reports also have suggested that non sulfated glycosaminoglycans such as Hyaluronan play a role in innate immunity. Evidence support that Hyaluronan degradation products transduce their inflammatory signal through TLR2, TLR 4 or both in macrophages and dendritic cells.

A large number of synthetic lipid A analogs have been prepared. Lien et. al described the agonist ER-112022, in which the disaccharide backbone of lipid A is replaced with —CH₂CH₂—NHCO—(CH₂)₄—CONH—(CH₂)₂. The two phosphate group link this substitute backbone to the lipid chains. (Lien E, Chow J C, Hawkins L D, McGuinness P D, Miyake K, Espevik T, Gusovsky F, Golenbock D T. A novel synthetic acyclic lipid A-like agonist activates cells via the lipopolysaccharide/toll-like receptor 4 signaling pathway. J Biol Chem. 2001; 276: 1873-1880.)

Christ et. al prepared the lipid A antagonist E5531 derived by modification of the structure of the endotoxin-antagonist Rhodobacter Capsulatus lipid A, in which naturally occurring acyl linkages at the C-3 and C-3′ carbons are replaced by ether linkages, and the C-6′ hydrozyl group was blocked which has resulted in increased stability and purity. (Christ W J, Asano O, Robidoux A L, Perez M, Wang Y, Dubuc G R, Gavin W E, Hawkins L D, McGuinness P D, Mullarkey M A, et al. E5531, a pure endotoxin antagonist of high potency. Science. 1995; 268:80-83.)

Qureshi et. al showed the minimal structure required for toxicity was a bisphosphorylated β-1,6-linked di glucosamine core to which long fatty acid chains are attached. The reports suggested that an optimal number of lipid chains in the form of acyl or acyloxyacyl groups are required on the disacharide backbone in order to exert strong endotoxic and related biological activities of Lipid A. (Qureshi N, Takayama K, Ribi E. Purification and structural determination of nontoxic lipid A obtained from the lipopolysaccharide of Salmonella typhimurium. J Biol Chem. 1982; 257: 11808-15; Kotani S, Takada H, Takahashi I, Ogawa T, Tsujimoto M, Shimauchi H, Ikeda T, Okamura H, Tamura T, Harada K, et al. Immunobiological activities of synthetic lipid A analogs with low endotoxicity. Infect Immun. 1986; 54: 673-82.)

Werner 1996 has suggested that removal of either phosphate group resulted in significant loss of toxicity without compromising on adjuvant activity. Bioassays on monophosphoryl Lipid A showed that although 1000 times less potent in eliciting toxic properties, it was comparable to diphoshoryl Lipid A in immunostimulating activities.

Further studies by Seydel and others suggested that the agonistic and antagonistic activity of Lipid A were governed by intrinsic conformation of Lipid A which in turn was defined by the number of charges, the number and distribution of acyl chains in the molecule. (Brandenburg K, Lindner B, Schromm A, Koch M H, Bauer J, Merkli A, Zbaeren C, Davies J G, Seydel U. Physicochemical characteristics of triacyl lipid A partial structure OM-174 in relation to biological activity. Eur J Biochem. 2000; 267: 3370-7; Schromm A B, Brandenburg K, Loppnow H, Moran A P, Koch M H, Rietschel E T, Seydel U. Biological activities of lipopolysaccharides are determined by the shape of their lipid A portion. Eur J Biochem. 2000; 267: 2008-13.)

Thus in view of the literature indicating synthetic lipid A derivatives behaving as TLR4 antagonists such as reported by Mullarkey et. al in relation to E5564 [α-D-glucopyranose, 3-O-decyl-2-deoxy-6-O-[2-deoxy-3-O-[(3R)-3-methoxydecyl]-6-O-methyl-2-[[(11Z)-1-oxo-11-octadecenyl]amino]-4-O-phosphono-β-D-glucopyranosyl]-2-[(1,3-dioxotetradecyl)amino]-1-(dihydrogen phosphate), tetrasodium salt] tetrasodium salt wherein the lipodisaccharide has a complicated structure posed more problems in synthesis and the chemical synthesis involves multisteps. (Mullarkey M, Rose J R, Bristol J, Kawata T, Kimura A, Kobayashi S, Przetak M, Chow J, Gusovsky F, Christ W J, Rossignol D P. Inhibition of endotoxin response by e5564, a novel Toll-like receptor 4-directed endotoxin antagonist. J Pharmacol Exp Ther. 2003; 304: 1093-102.) It is well documented that most known TLR ligands contain carbohydrate moieties and the potential role of pure carbohydrates or its analogues as ligands for TLRs has unlimited scope. Also, documented reports observe low molecular weight hyaluronic acid oligosaccharides produced during inflammation exhibiting ability to induce maturation of DCs through TLR4. (Termeer C, Benedix F, Sleeman J, Fieber C, Voith U, Ahrens T, Miyake K, Freudenberg M, Galanos C, Simon J C. Oligosaccharides of Hyaluronan activate dendritic cells via toll-like receptor 4. J Exp Med. 2002; 195: 99-111; Monari C, Bistoni F, Casadevall A, Pericolini E, Pietrella D, Kozel T R, Vecehiarelli A. Glucuronoxylomannan, a microbial compound, regulates expression of costimulatory molecules and production of cytokines in macrophages. J Infect Dis. 2005; 191: 127-37.)

### SUMMARY OF THE INVENTION

It is the object of the present invention to provide carbohydrate based molecules for modulation of immunity.

It is the object of the present invention to provide carbohydrate based molecules for inhibition of TLR ligands.

It is the object of the present invention to provide methods of preparation of the carbohydrate based molecules.

It is the object of the present invention to provide compositions of the carbohydrate based molecules.

It is the object of the present invention to provide compositions useful for the treatment of prevention or treatment of inflammation, wounds, autoimmunity, allergy, asthma, graft rejection, graft versus host disease, infection, sepsis, cancer, and immunodeficiency.

It is the object of the present invention to provide compositions for inhibition of TLR mediated conditions.

It is the object of the present invention to provide carbohydrate based molecules for inhibition of TLR ligands which can be used in combinations with other agents.

Applicants have developed alternate synthetic TLR4 antagonists for the effective treatment of TLR4 ligand/signaling/LPS associated disorders and which can be used as monotherapies in the absence of antigen.

The present invention provides novel carbohydrate based disaccharides for modulation of immunity with substantial antagonistic activity in vitro, wherein efficient and selective inhibition of TLR-mediated production of TNF-α occurs. Further, mRNA expression of various pro-inflammatory genes as a result of NF-κB activation and in vivo systems where it significantly reduced LPS-induced TNF in mice and Carrageenan induced footpad edema in mice.

The present invention relates to carbohydrate-based molecules, methods of preparations, compositions for use in TLR mediated immune conditions. The present invention also relates to compositions and methods for modulating immune functions mediated through Toll-like receptor (TLR) for efficient inhibition of TLR4 ligand mediated signaling events and consequences. Moreover, further studies on the present invention provides possibilities of monotherapies or combinations thereof formulated and administered in the absence of exogenous antigens for the therapeutic/prophylactic treatment of the plant and animal diseases.

In one embodiment the present invention provides compositions that are useful for the prevention or treatment of inflammation, wounds, autoimmunity, allergy, asthma, graft rejection, graft versus host disease, infection, sepsis, cancer, and immunodeficiency. In the preferred embodiments the compositions as described in the present invention are useful for inhibition of TLR signaling in response to TLR ligands.

In the preferred embodiments the present invention provides the compositions for inhibition of TLR signaling in a therapeutically effective amount and pharmaceutically inert adjuvants, diluents or carriers.

In one embodiment the compositions as described in the present invention or composition comprising the same is believed to have the ability to inhibit inhibition of TLR signaling under physiological conditions, and thereby would have corresponding effectiveness for prevention or treatment of inflammation, wounds, autoimmunity, allergy, asthma, graft rejection, graft versus host disease, infection, sepsis, cancer, and immunodeficiency.

In yet another embodiment on basis of inhibition of TLR signaling properties, the composition can be used in veterinary medicine for the prevention and treatment of inflammation, wounds, autoimmunity, allergy, asthma, graft rejection, graft versus host disease, infection, sepsis, cancer, and immunodeficiency.

In preferred embodiments the present invention also provides the pharmaceutical formulations either alone or a suitable pharmaceutically acceptable adjuvant useful in inhibition of TLR mediated clinical manifestations.

The compositions as described in the present invention are useful for the prevention or treatment of inflammation, wounds, autoimmunity, allergy, asthma, graft rejection, graft versus host disease, infection, sepsis, cancer, and immunodeficiency. As a feature of the present invention, the methods of the invention can be combined with administration of additional agents to achieve synergistic effect on TLR-mediated immunostimulation. More specifically, whereas the agents described herein have been discovered to affect TLRs directly and thus directly affect TLR-bearing cells, e.g., antigen-presenting cells (APCs), such agents can be used in conjunction with additional agents which affect non-APC immune cells, e.g., T lymphocytes (T cells). Such an approach effectively introduces an immunomodulatory intervention at two levels: innate immunity and acquired immunity. Since innate immunity is believed to initiate and support acquired immunity, the combination intervention is synergistic

The present invention also provides the manner of manufacture of compositions as described in the present invention in a therapeutically effective amount either alone or in combination with pharmaceutically acceptable adjuvant.

In another embodiment of the invention, a method of affecting TLR-mediated signaling in response to a TLR ligand is provided.

In one embodiment of the invention, a method of inhibiting TLR-mediated immunostimulatory signaling is provided.

In another embodiment, the invention provides a method of modulating TLR-mediated immunostimulation in a subject.

The carbohydrate-based molecule of the present invention can be used in the treatment for variety of conditions involving autoimmunity, inflammation, allergy, asthma, graft rejection, graft-versus-host disease (GvHD), infection, sepsis, cancer, and immunodeficiency. Generally, for treating conditions involving infection, cancer, and immunodeficiency employs small molecules that augment TLR-mediated signaling in response to a suitable TLR ligand. In some instances the methods can be used to inhibit or promote TLR-mediated signaling in response to a TLR ligand or TLR signaling agonist. In some instances the methods can be used to inhibit TLR-mediated immunostimulatory signaling in response to a TLR ligand or TLR signaling agonist. In some instances the methods can be used to inhibit or promote TLR-mediated immunostimulation in a subject. In some instances the methods can be used to inhibit TLR-mediated immunostimulation in a subject. In some instances the methods can be used to inhibit an immunostimulatory nucleic acid-associated response in a subject.

In one embodiment, the present invention provides molecules and methods useful for modulating TLR-mediated signaling. The molecules of the present invention are applicable to alter any TLR mediated signaling in response to a suitable ligand or signaling agonist.

In one embodiment the present invention also provides methods for identifying agents that decrease or inhibit activation of Toll-like receptor 2. These methods involve (i) contacting a cell expressing the receptor with a candidate agent in the presence of an activator of the receptor (in vitro or in vivo) and (ii) determining the effect of the agent on activation of the receptor. Detection of a decrease in activation of the receptor by the activator in the presence of the agent indicates the identification of agent that can be used to decrease or inhibit activation of the receptor. In these methods, the effect of the agent on the activation of the receptor can be determined by analysis of the expression of a reporter gene that is under the control of a promoter that is induced in a signaling pathway triggered by activation of the receptor.

In one aspect of the invention, a method of affecting TLR-mediated signaling in response to a TLR ligand is provided. The method according to this aspect involves contacting a cell expressing a TLR with an effective amount of a compound of
Formula I: β-D-glucopyranosyl-(1-3)-2-deoxy-2-amino-α-D-glucopyranoside

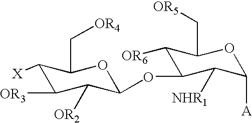

wherein all or at least one R1, R2, R3 or R4 is;

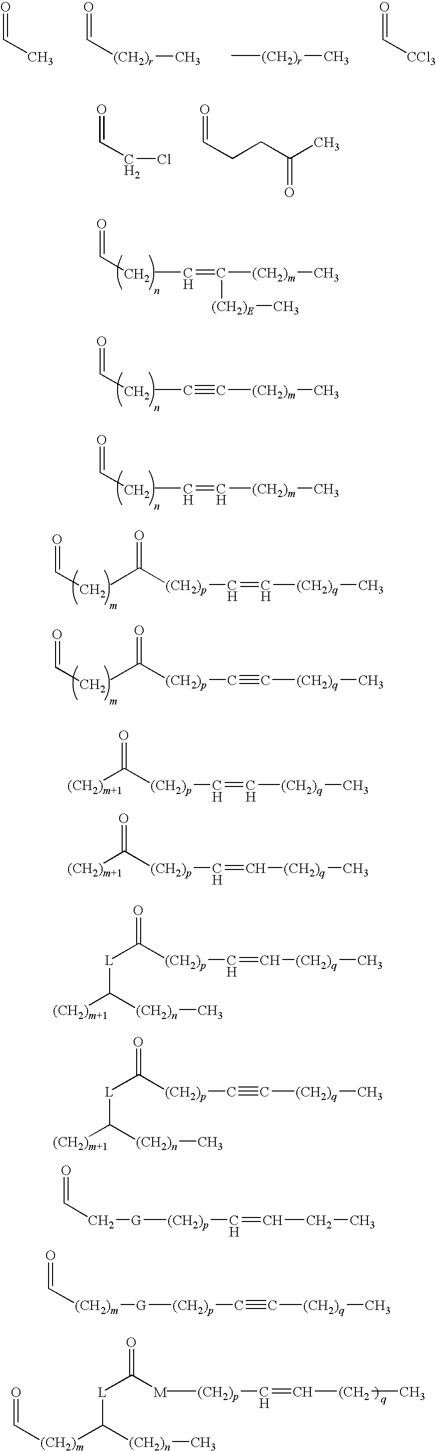

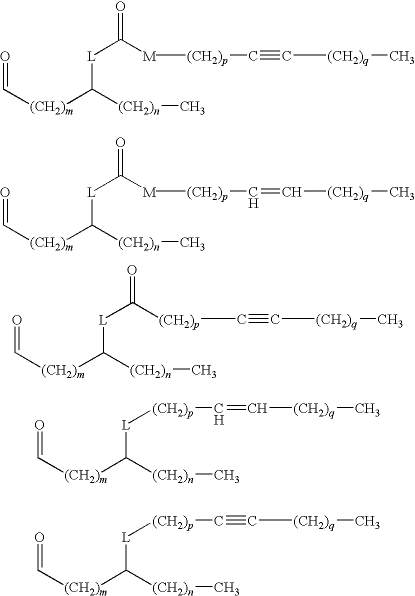

wherein each L is O, N or C; each M is O or N;

each E, independently, is an integer between 0 and 14 inclusive;

each G, independently, is N, O, S, SO, or SO₂;

each m, independently, is an integer between 0 and 14 inclusive;

each n, independently, is an integer between 0 and 14 inclusive;

each p, independently, is an integer between 0 and 10 inclusive;

each r, independently is an integer between 0 and 20 inclusive; and

each q, independently, is an integer between 0 and 10 inclusive;

each of the remaining R1, R2, R3, and R4, independently, is:

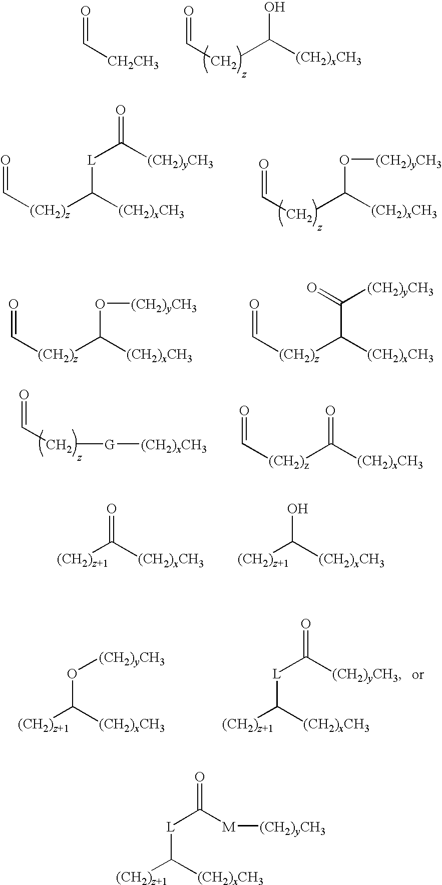

wherein each L is O, N or C;

each M is O or N;

each x, independently, is an integer between 0 and 14 inclusive;

each y, independently, is an integer between 0 and 14 inclusive;

each z, independently, is an integer between 0 and 10 inclusive;

each G, independently, is N, O, S, SO, SO₂; and

each A and X, independently, is H, OH, OCH₃, C₆H₅OCH₃,

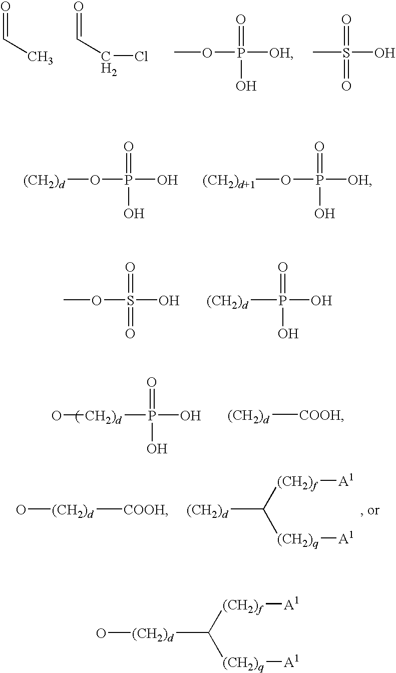

wherein each d, independently, is an integer between 0 and 5 inclusive;

each f, independently, is an integer between 0 and 5 inclusive;

each g, independently, is an integer between 0 and 5 inclusive; and

each A¹, independently, is

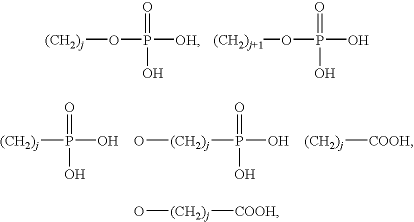

wherein each j, independently, is an integer between 0 and 14 inclusive;

X is OH, Cl, O(CH₂)ₜCH₃, O(CH₂)ₜOH, O(CH₂)ₜO(CH₂)ᵥCH₃

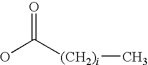

wherein i, independently, is an integer between 0 and 20 inclusive;

each t and v, independently, is an integer between 0 and 14 inclusive;

R₅ and R₆ are any possibilities listed above for R₁-R₄ and in addition R₅ and R₆ are H, benzylidene, acetonide;

Preferably, the above compounds are formulated as a lysine salt, a TRIS salt, a potassium or a sodium salt.

In a second aspect, the invention features a compound of the formula:
Formula II: 2-Deoxy-2-amino-β-D-glucopyranosyl-(1-4)-β-D-glucopyranoside

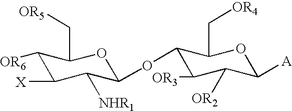

wherein all or at least one R1, R2, R3 or R4 is;

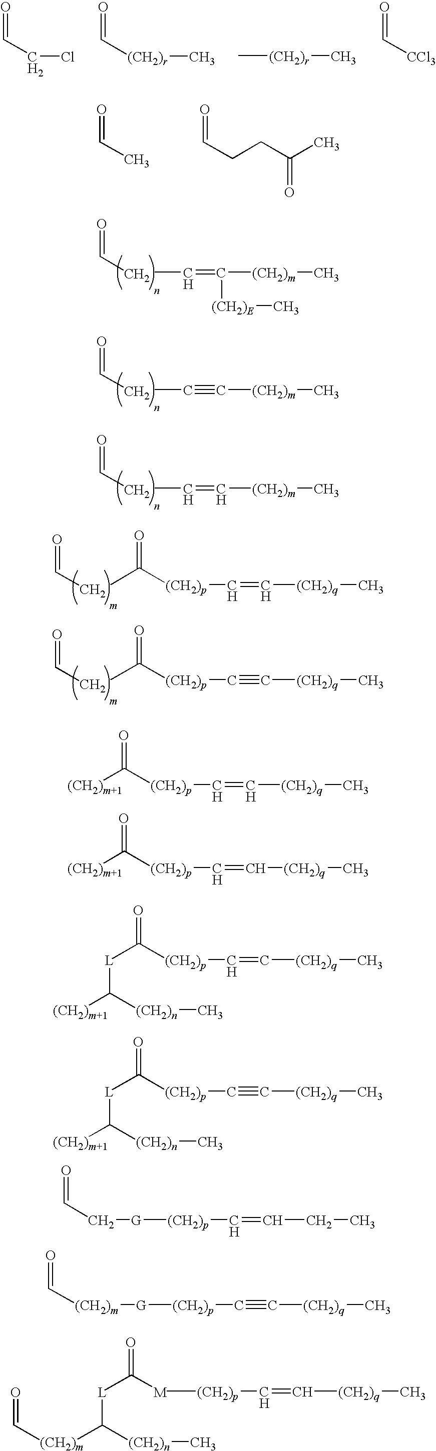

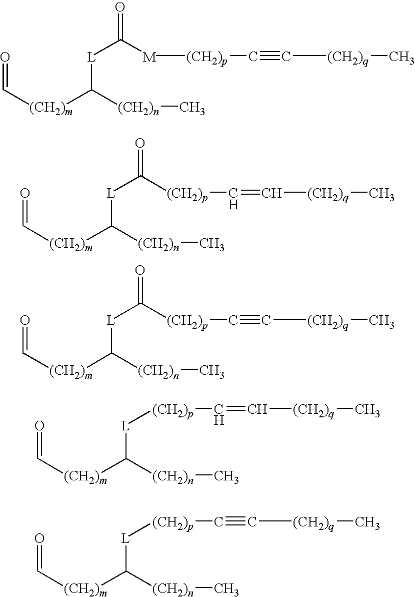

wherein each L is O, N or C;

each M is O or N;

each E, independently, is an integer between 0 and 14 inclusive;

each G, independently, is N, O, S, SO, or SO₂;

each m, independently, is an integer between 0 and 14 inclusive;

each n, independently, is an integer between 0 and 14 inclusive;

each p, independently, is an integer between 0 and 10 inclusive;

each r, independently, is an integer between 0 and 20 inclusive; and

each q, independently, is an integer between 0 and 10 inclusive;

each of the remaining R1, R2, R3, and R4, independently, is:

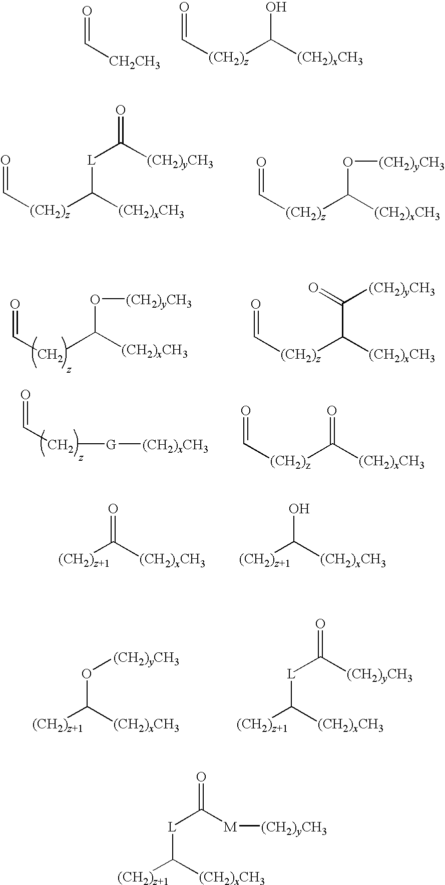

wherein each L is O, N or C;

each M is O or N;

each x, independently, is an integer between 0 and 14 inclusive;

each y, independently, is an integer between 0 and 14 inclusive;

each z, independently, is an integer between 0 and 10 inclusive; and

each G, independently, is N, O, S, SO, SO₂;

each A and X, independently, is H, OH, OCH₃, C₆H₅OCH₃,

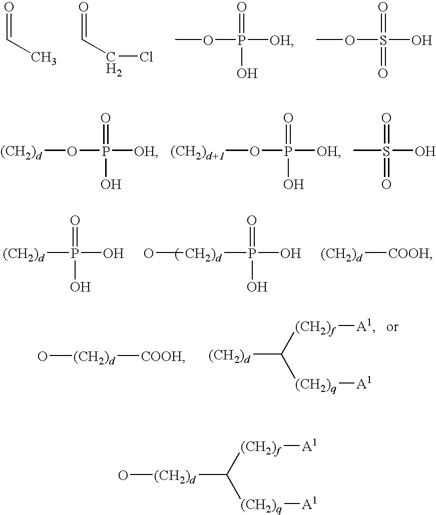

wherein each d, independently, is an integer between 0 and 5 inclusive;

each f, independently, is an integer between 0 and 5 inclusive;

each g, independently, is an integer between 0 and 5 inclusive and

each A¹, independently, is

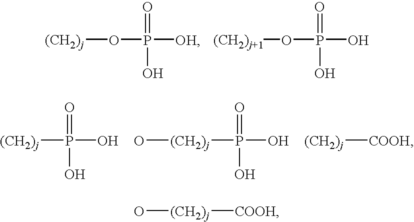

wherein each j, independently, is an integer between 0 and 14 inclusive;

X is OH, Cl, O(CH₂)ₜCH₃, O(CH₂)ₜOH, O(CH₂)ₜO(CH₂)ᵥCH₃

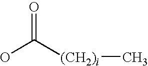

wherein i, independently, is an integer between 0 and 20 inclusive;

each t and v, independently, is an integer between 0 and 14 inclusive;

R₅ and R₆ is any possibilities listed above for R₁-R₄ and in addition, R₅ and R₆ are H, benzylidene, acetonide.

Most preferably, the above compounds are formulated as a lysine salt, a TRIS salt, a potassium salt or a sodium salt.

In a third aspect, the invention features a compound of the formula:
Formula III: 2-Deoxy-2-amino-β-D-glucopyranosyl-(1-4)-O-(β-D-glucopyranosyl)-(1-3)-α-2-deoxy-2-amino-D-glucopyranoside

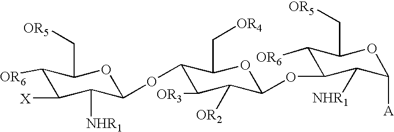

wherein all or at least one of R1, R2, R3 or R4 is:

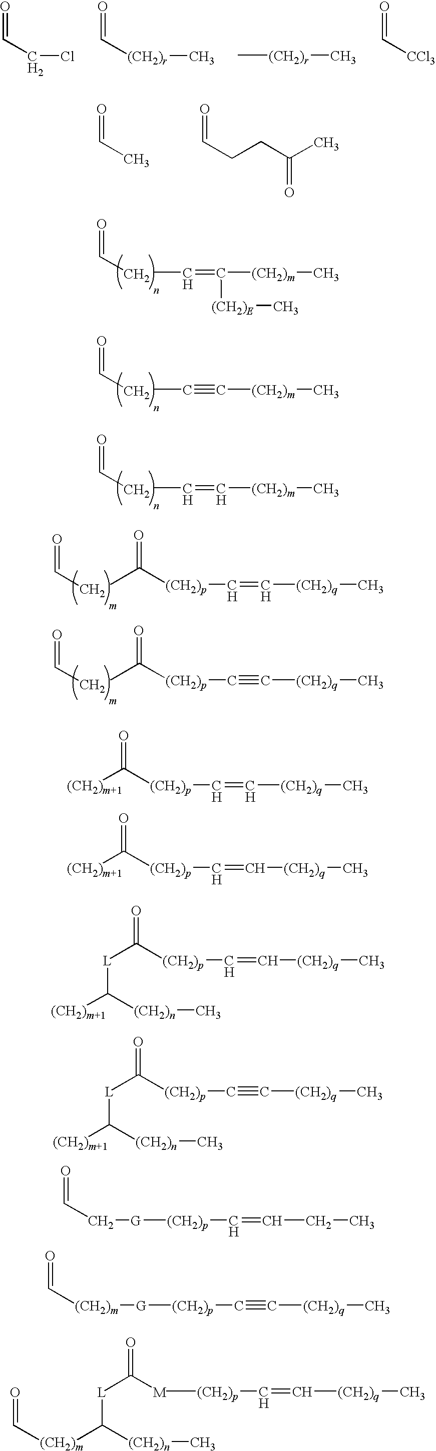

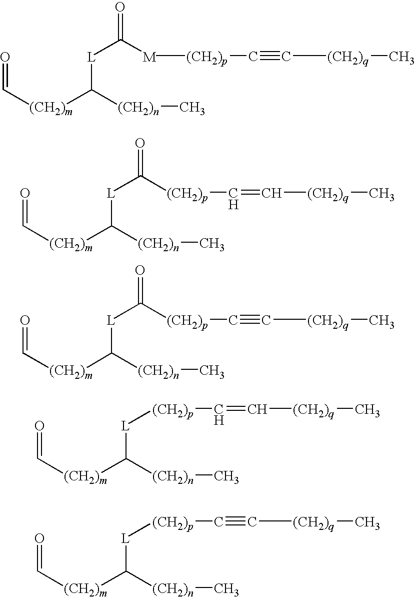

wherein each L is O, N or C; each M is O or N;

each E, independently, is an integer between 0 and 14 inclusive;

each G, independently, is N, O, S, SO, or SO₂;

each m, independently, is an integer between 0 and 14 inclusive;

each n, independently, is an integer between 0 and 14 inclusive;

each p, independently, is an integer between 0 and 10 inclusive;

each r, independently, is an integer between 0 and 20 inclusive and

each q, independently, is an integer between 0 and 10 inclusive;

each of the remaining R1, R2, R3, and R4, independently, is:

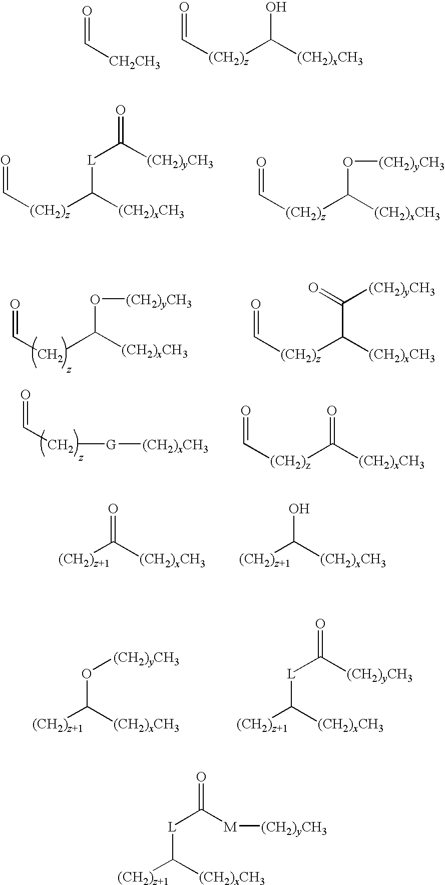

wherein each L is O, N or C; each M is O or N;

each x, independently, is an integer between 0 and 14 inclusive;

each y, independently, is an integer between 0 and 14 inclusive;

each z, independently, is an integer between 0 and 10 inclusive and

each G, independently, is N, O, S, SO, SO₂;

each A and X, independently, is H, OH, OCH₃, C₆H₅OCH₃,

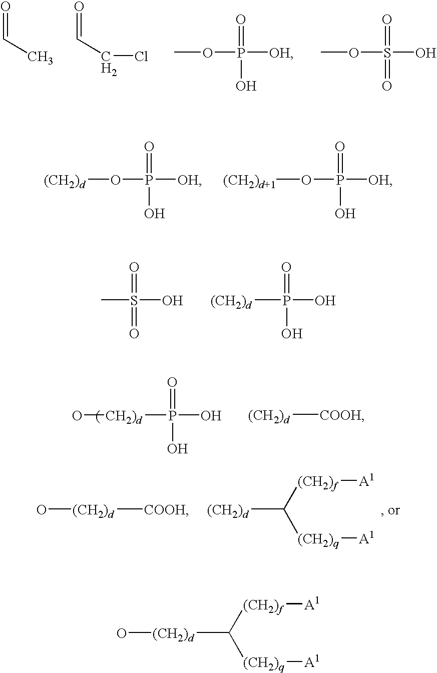

wherein each d, independently, is an integer between 0 and 5 inclusive;

each f, independently, is an integer between 0 and 5 inclusive;

each g, independently, is an integer between 0 and 5 inclusive and

each A¹, independently, is

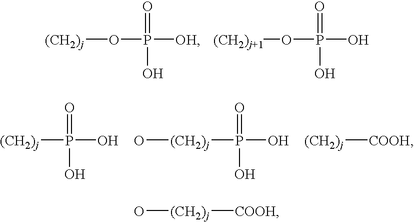

wherein each j, independently, is an integer between 0 and 14 inclusive;

X is OH, Cl, O(CH₂)ₜCH₃, O(CH₂)ₜOH, O(CH₂)ₜO(CH₂)ᵥCH₃

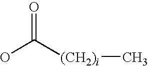

wherein i, independently, is an integer between 0 and 20 inclusive;

each t and v, independently, is an integer between 0 and 14 inclusive;

R₅ and R₆ is any possibilities listed above for R₁-R₄, and in addition R₅ and R₆ are H, benzylidene, acetonide;

Preferably, the above compounds are formulated as a lysine salt, a TRIS salt, a potassium salt or a sodium salt.

In a fourth aspect, the invention features a compound of the formula:
Formula IV: (β-D-Glucopyranosyl-(1-3)-O-(2-deoxy-2-amino-β-D-glucopyranosyl)-(1-4)-β-D-glucopyranoside

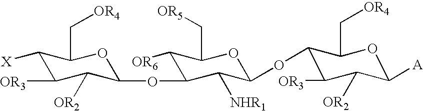

wherein all or at least one R1, R2, R3 or R4 is;

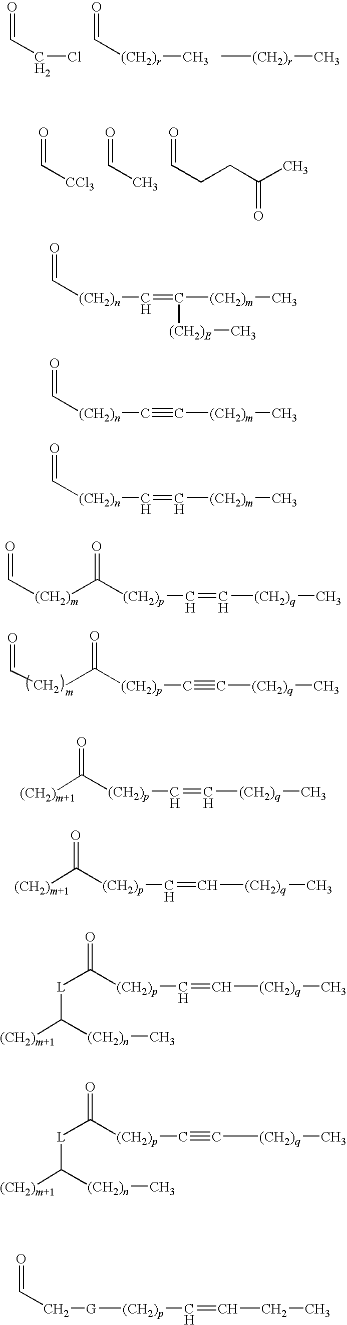

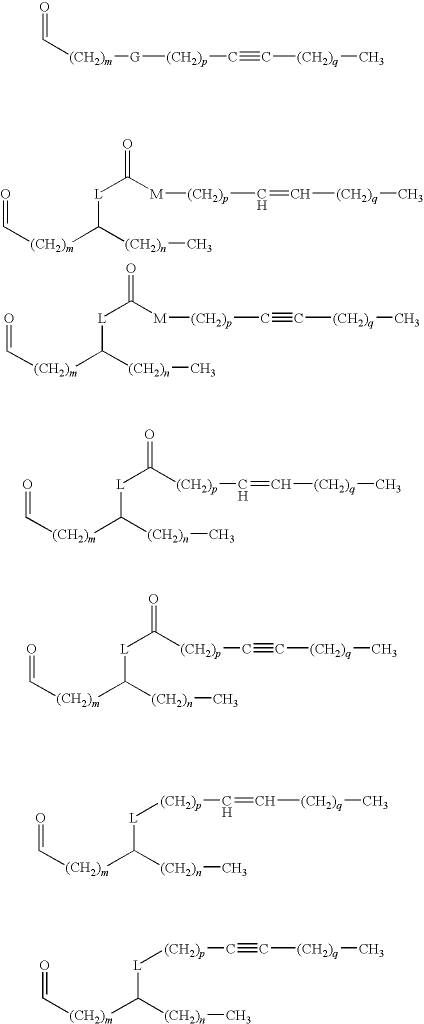

wherein each L is O, N or C; each M is O or N;

each E, independently, is an integer between 0 and 14 inclusive;

each C, independently, is N, O, S, SO, or SO₂;

each m, independently, is an integer between 0 and 14 inclusive;

each n, independently, is an integer between 0 and 14 inclusive;

each p, independently, is an integer between 0 and 10 inclusive;

each r, independently, is an integer between 0 and 20 inclusive and

each q, independently, is an integer between 0 and 10 inclusive;

each of the remaining R1, R2, R3, and R4, independently, is:

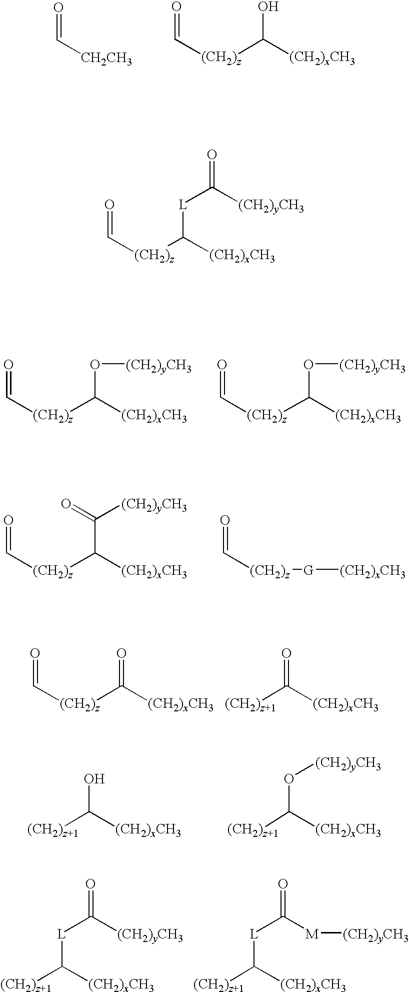

wherein each L is O, N or C; each M is O or N;

each x, independently, is an integer between 0 and 14 inclusive;

each y, independently, is an integer between 0 and 14 inclusive;

each z, independently, is an integer between 0 and 10 inclusive and

each G, independently, is N, O, S, SO, SO₂;

each A and X, independently, is H, OH, OCH₃, C₆H₅OCH₃,

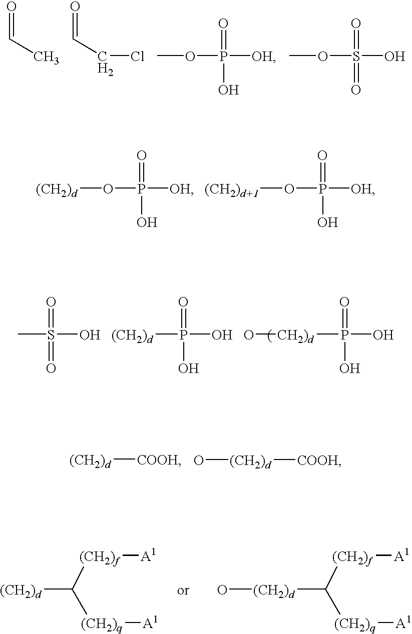

wherein each d, independently, is an integer between 0 and 5 inclusive;

each f, independently, is an integer between 0 and 5 inclusive;

each g, independently, is an integer between 0 and 5 inclusive and

each A¹, independently, is

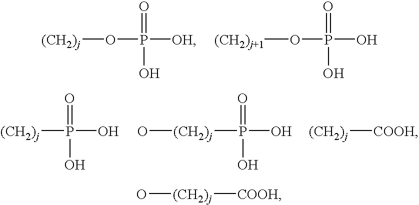

wherein each j, independently, is an integer between 0 and 14 inclusive;

X is OH, Cl, O(CH₂)ₜCH₃, O(CH₂)ₜOH, O(CH₂)ₜO(CH₂)ᵥCH₃

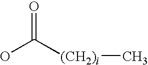

wherein i, independently, is an integer between 0 and 20 inclusive;

each t and v, independently, is an integer between 0 and 14 inclusive;

R₅ and R₆ is any possibilities listed above for R₁-R₄, in addition, R₅ and R₆ are H, benzylidene, acetonide;

Preferably, the above compounds are formulated as a lysine salt, a TRIS salt, a potassium salt or a sodium salt.

In a fifth aspect, the invention features a compound V of the formula:
Formula V: (β-D-Glucopyranosyl)-(1-3)-O-(2-deoxy-2-amino-β-D-glucopyranosyl)-(1-4)-O-(β-D-glucopyranosyl)-(1-3)-O-2-deoxy-2-amino-α-D-glucopyranoside

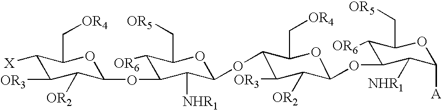

wherein at least one R1, R2, R3 or R4 is;

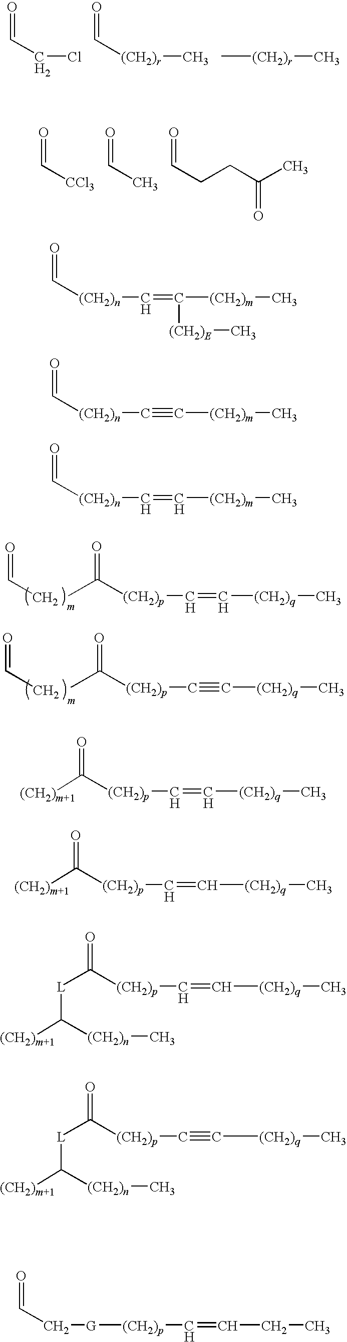

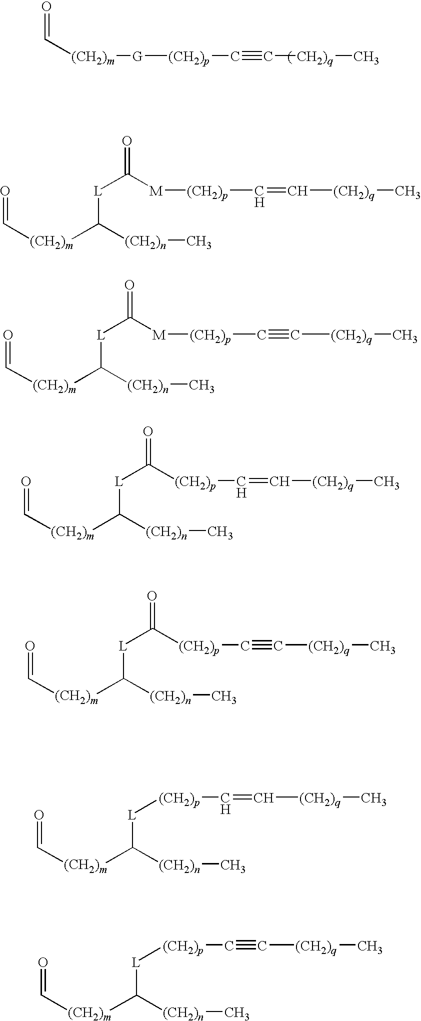

wherein each L is O, N or C; each M is O or N;

each E, independently, is an integer between 0 and 14 inclusive;

each G, independently, is N, O, S, SO, or SO₂;

each m, independently, is an integer between 0 and 14 inclusive;

each n, independently, is an integer between 0 and 14 inclusive;

each p, independently, is an integer between 0 and 10 inclusive;

each r, independently, is an integer between 0 and 20 inclusive and

each q, independently, is an integer between 0 and 10 inclusive;

each of the remaining R1, R2, R3, and R4, independently, is:

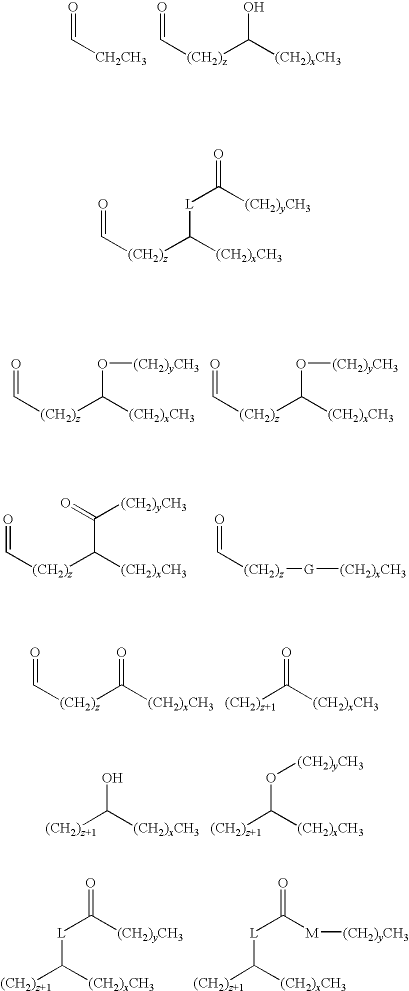

wherein each L is O, N or C; each M is O or N;

each x, independently, is an integer between 0 and 14 inclusive;

each y, independently, is an integer between 0 and 14 inclusive;

each z, independently, is an integer between 0 and 10 inclusive and

each G, independently, is N, O, S, SO, SO₂;

each A and X, independently, is H, OH, OCH₃, C₆H₅OCH₃,

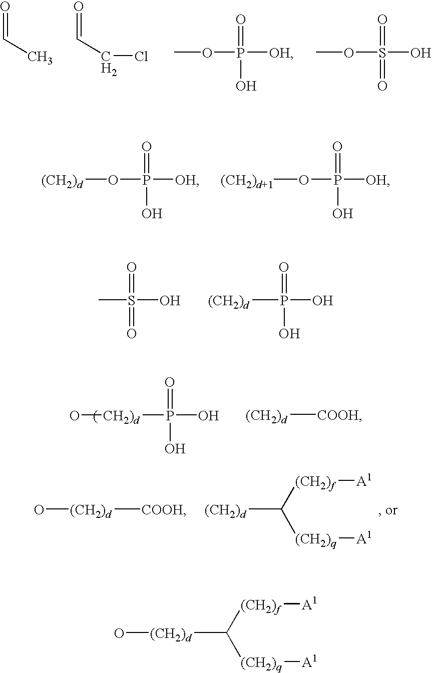

wherein each d, independently, is an integer between 0 and 5 inclusive;

each f, independently, is an integer between 0 and 5 inclusive;

each g, independently, is an integer between 0 and 5 inclusive and

each A¹, independently, is

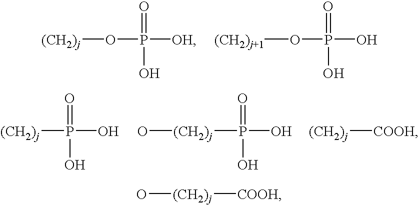

wherein each j, independently, is an integer between 0 and 14 inclusive;

X is OH, Cl, O(CH₂)ₜCH₃, O(CH₂)ₜOH, O(CH₂)ₜO(CH₂)ᵥCH₃

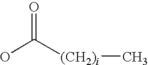

wherein i, independently, is an integer between 0 and 20 inclusive;

each t and v, independently, is an integer between 0 and 14 inclusive;

R₅ and R₆ is any possibilities listed above for R₁-R₄, in addition, R₅ and R₆ are H, benzylidene, acetonide;

Preferably, the above compounds are formulated as a lysine salt, a TRIS salt, a potassium salt or a sodium salt.

In a sixth aspect, the invention features a compound of the formula:
Formula VI: (2-Deoxy-2-amino-β-D-glucopyranosyl)-(1-4)-O-(β-D-glucopyranosyl)-(1-3)-O-(2-deoxy-2-amino-β-D-glucopyranosyl)-(1-4)-O-β-D-glucopyranoside

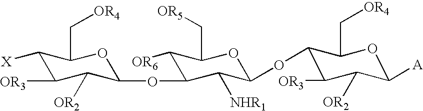

wherein all or at least one R1, R2, R3 or R4 is;

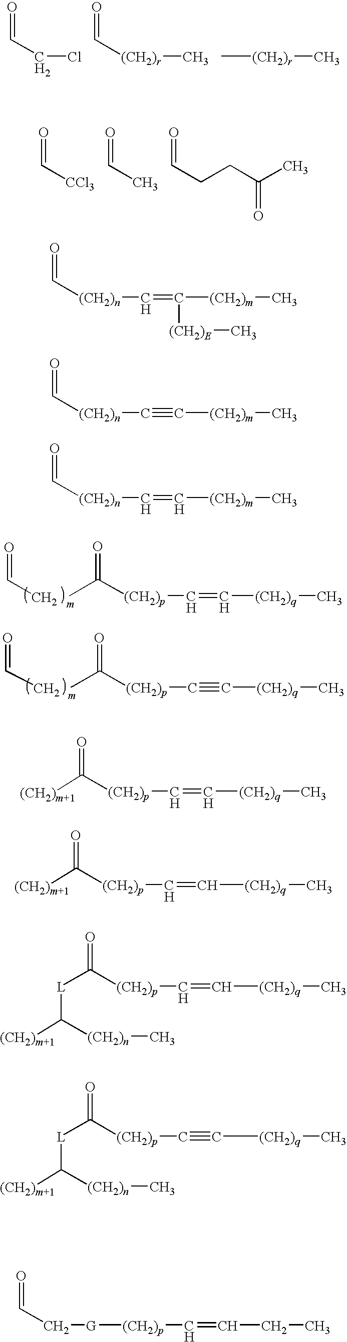

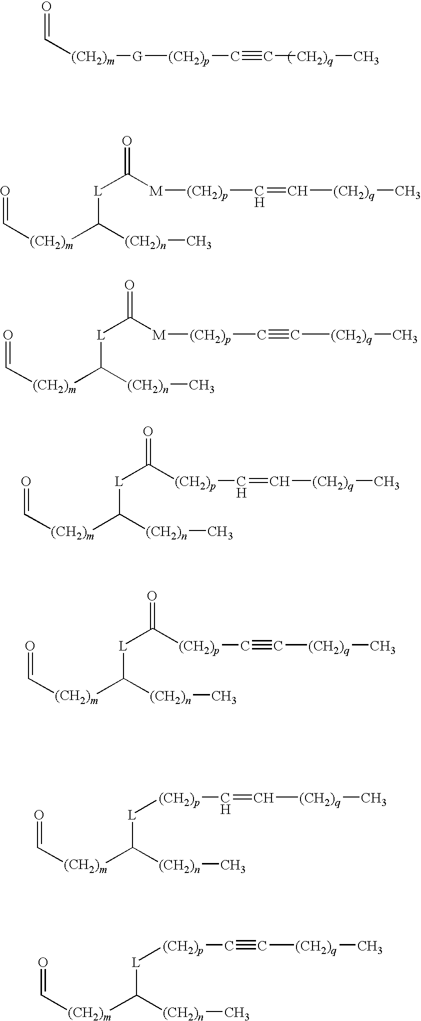

wherein each L is O, N or C; each M is O or N;

each E, independently, is an integer between 0 and 14 inclusive;

each G, independently, is N, O, S, SO, or SO₂;

each m, independently, is an integer between 0 and 14 inclusive;

each n, independently, is an integer between 0 and 14 inclusive;

each p, independently, is an integer between 0 and 10 inclusive;

each r, independently, is an integer between 0 and 20 inclusive and

each q, independently, is an integer between 0 and 10 inclusive;

each of the remaining R1, R2, R3, and R4, independently, is:

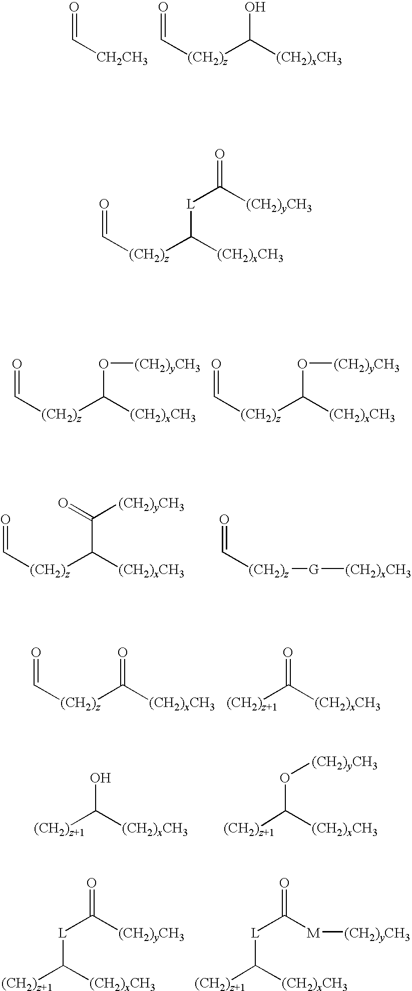

wherein each L is O, N or C; each M is O or N;

each x, independently, is an integer between 0 and 14 inclusive;

each y, independently, is an integer between 0 and 14 inclusive;

each z, independently, is an integer between 0 and 10 inclusive and

each G, independently, is N, O, S, SO, SO₂;

each A and X, independently, is H, OH, OCH₃, C₆H₅OCH₃,

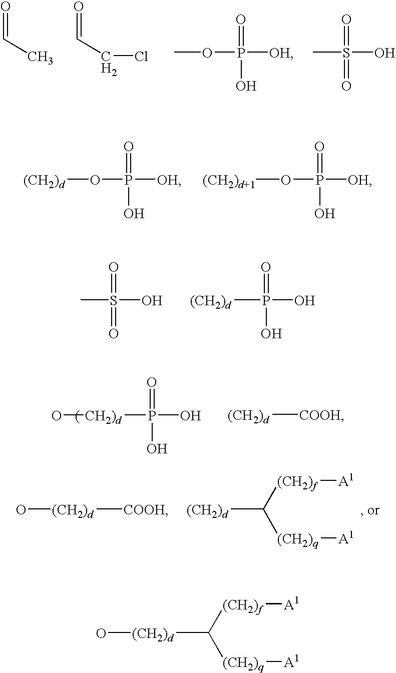

wherein each d, independently, is an integer between 0 and 5 inclusive;

each f, independently, is an integer between 0 and 5 inclusive;

each g, independently, is an integer between 0 and 5 inclusive and

each A¹, independently, is

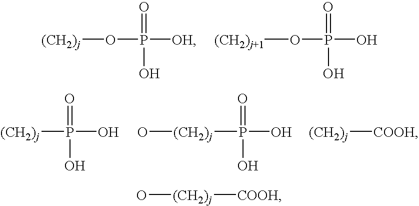

wherein each j, independently, is an integer between 0 and 14 inclusive;

X is OH, Cl, O(CH₂)ₜCH₃, O(CH₂)ₜOH, O(CH₂)ₜO(CH₂)ᵥCH₃

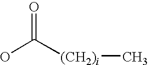

wherein i, independently, is an integer between 0 and 20 inclusive;

each t and v, independently, is an integer between 0 and 14 inclusive;

R₅ and R₆ is any possibilities listed above for R₁-R₄, in addition R₅ and R₆ are H, benzylidene, acetonide.

Preferably, the above compounds are formulated as a lysine salt, a TRIS salt, a potassium or a sodium salt.

Accordingly, the invention features compounds of formula I-IV and a pharmaceutically acceptable salt or prodrug thereof.

### BRIEF DESCRIPTION OF THE FIGURES

The following figures form part of the present specification and are included to further demonstrate certain aspects of the present disclosure, the inventions of which can be better understood by reference to one or more of these drawings in combination with the detailed description of specific embodiments presented herein.

FIG. 1a shows that RSCL-0409 inhibits TLR2 and TLR4 induced TNF-α secretion in human monocytic (THP-1) cells.

FIG. 1b shows that RSCL-0409 inhibits TLR2 and TLR4 induced TNF-α secretion in and peripheral blood monocytes (PBMCs).

FIG. 2a shows inhibition of TNF-α secretion in THP-1 cells by RSCL-0409 is dose-dependent

FIG. 2b indicates that RSCL-0409 is not toxic to THP-1 cells

FIG. 3 shows RSCL-0409 inhibits LPS induced TNF-α release in PBMC

FIG. 4 shows the dose dependent effect of RSCL-0409 on different concentrations of LPS in THP-1 cells

FIG. 5a shows effect of RSCL-0409 on TNF-α mRNA expression in THP-1 cells in real time

FIG. 5b shows effect of RSCL-0409 on IL-6 mRNA expression in THP-1 cells in real time

FIG. 5c shows effect of RSCL-0409 on mRNA expression of pro-inflammatory genes in THP-1 Cells

FIG. 5d Demonstrates ability of RSCL-0409 to inhibit Arachadonic acid induced PGE2 release in A549 cells

FIG. 6 shows RSCL-0409 suppresses LPS induced Nitric oxide (NO) release in RAW 264.7 cells

FIGS. 7a, 7b and 7c show RSCL-0409 blocking activation of NEMO, degradation of IκB-α and activation of NF-κB.

FIG. 7a represents effect of RSCL-0409 on activation of NEMO and degradation of I kappa B-alpha (IκB-α).

FIG. 7b represents effect of RSCL-0409 activation of NF-κB.

FIG. 7c represents effect of RSCL-0409 on translocation of NF-κB to the nucleus

FIG. 8 shows effect of RSCL-0409 on NF-κB activation in secreted embryonic alkaline phosphatase (SEAP) reporter assay.

FIGS. 9a and 9b show the effect of RSCL-0409 on MyD88 dependent TLR signaling

FIG. 9a represents effect of RSCL-0409 on TLR related genes

FIG. 9b represents RSCL-0409 inhibits MyD88 dependent TLR signaling by LPS.

FIGS. 10a and 10b show pre-treatment of RSCL-0409 as well as post LPS treatment suppresses induced TNF-α release in Balb/c mice

FIG. 10a shows pre-treatment of RSCL-0409 suppresses LPS induced TNF-α release in Balb/c mice

FIG. 10b Treatment with RSCL-0409 post LPS induction suppresses the induced TNF-α release in Balb/c mice

### DETAILED DESCRIPTION OF THE INVENTION

### Definitions

The term “carbohydrate based molecules” as used herein refers to molecules with basic carbohydrate backbone in pyranose (six membered) configuration linked through a glycosidic bond.

The term “LPS” as used herein refers to Lipopolysaccharide. Lipopolysaccharide (LPS), which is contained in the outer membrane of the cell wall of various gram-negative bacteria, consists of a glycolipid called “Lipid A” to which various saccharides are bonded. It has been known for along time that LPS is the main component of endotoxins.

The term “TLR” as used herein refers to Toll like receptor.

The term “pharmaceutically acceptable salt,” as use herein, refers to those salts which are, within the scope of sound medical, suitable for use in contact with the tissues of humans and animals without undue toxicity, irritation, allergic response and the like and are commensurate with a reasonable benefit/risk ratio. Pharmaceutically acceptable salts are well known in the art. For example, S. M. Berge et al. describe pharmaceutically acceptable salts in detail in J. Pharmaceutical Sciences 66:119, 1977. The salts can be prepared in situ during the final isolation and purification of a compound of the invention or separately by reacting the free base group with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The term “pharmaceutically acceptable ester,” as used herein, represents esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic, and alkanedioic acids, in which each alkyl or alkenyl group preferably has not more than 6 carbon atoms. Examples of particular esters include formates, acetates, propionates, butyates, acrylates, and ethylsuccinates. The term “pharmaceutically acceptable prodrugs,” as used herein, means prodrugs of the compounds of the present invention which are, within the scope of sound medical judgement, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “prodrug,” as used herein, represents compounds that are transformed in vivo into a parent compound of the above formula, for example, by hydrolysis in blood. A thorough discussion of prodrugs is provided in T. Higuchi and V. Stella, “Pro-drugs as Novel Delivery Systems,” Vol. 14 of the A.C.S. Symposium Series, Edward B. Roche, ed., “Bioreversible Carriers in Drug Design,” American Pharmaceutical Association and Pergamon Press, 1987, and Judkins et al., Synthetic Communications 26(23):43514367, 1996, each of which is incorporated herein by reference.

Asymmetric or chiral centers may exist in the compounds of the present invention. The present invention includes the various stereoisomers and mixtures thereof. Individual stereoisomers of compounds or the present invention may be prepared synthetically from commercially available starting materials that contain asymmetric or chiral centers or by preparation of mixtures of enantiometic compounds followed by resolution well-known to those of ordinary skill in the art. These methods of resolution are exemplified by (1) attachment of a racemic mixture of enantiomers, designated (+/−), to a chiral auxiliary, separation of the resulting diastereomers by recrystallization or chromatography and liberation of the optically pure product from the auxiliary or (2) direct separation of the mixture of optical enantiomers on chiral chromatographic columns. Enantiomers are designated herein by the symbols “R” or “S,” depending on the configuration of substituents around the chiral carbon atom, or are drawn by conventional means with a bolded line defining a substituent above the plane of the page in three-dimensional space and a hashed or dashed line defining a substituent beneath the plane of the printed page in three-dimensional space. If no stereochemical designation is made, it is to be assumed that the structure definition includes both stereochemical possibilities.

All of the starting materials used in any of these methods are commercially available from chemical vendors such as Aldrich, Sigma, Nova Biochemicals, Bachem Biosciences, Advanced ChemTech, and the like, or may be readily synthesized by known procedures.

The reaction products are isolated and purified by conventional methods, typically by solvent extraction into a compatible solvent. The products may be further purified by column chromatography or other appropriate methods, including medium pressure or high pressure liquid chromatography.

The compounds and methods of the invention are described in further detail, as follows:

### Therapeutic Use of TLR Antagonist

The present invention provides agents that can be used to prevent or to treat LPS mediated diseases or conditions that are characterized by TLR activation. The conditions that are prevented or treated but are not limited to inflammation, wounds, autoimmunity, allergy, asthma, graft rejection, graft versus host disease, infection, sepsis, cancer, and immunodeficiency.

Delivery and Dosage of the TLR Antagonist:

The present invention provides compositions comprising carbohydrate based molecules in an effective amount that achieves the desired therapeutic effect for a particular condition, patient and mode of administration. The dosage level selected depends on the route of administration and the severity of the condition being treated.

For example: For adults, the doses are generally from about 0.01 to about 100 mg/kg, desirably about 0.1 to about 1 mg/kg body weight per day by inhalation, from about 0.01 to about 100 mg/kg, desirably 0.1 to 70 mg/kg, more desirably 0.5 to 10 mg/kg body weight per day by oral administration, and from about 0.01 to about 50 mg/kg, desirably 0.1 to 1 mg/kg body weight per day by intravenous administration. Doses are determined for each particular case using standard methods in accordance with factors unique to the patient, including age, weight, general state of health, and other factors that can influence the efficacy of the compound(s) of the invention.

Further the administration of the compounds of the present invention is not limited to mammal, including humans, be limited to a particular mode of administration, dosage, or frequency of dosing.

The present invention encompasses all modes of administration, including oral, intraperitoneal, intramuscular, intravenous, intra-articular, intralesional, subcutaneous, or nasally, rectally, buccally, or any other route sufficient to provide a dose adequate to prevent or treat excess or undesired TLR activity.

The present invention also contemplates that one or more compounds may be administered to a mammal in a single dose or multiple doses. When multiple doses are administered, the doses may be separated from one another by, for example, several hours, one day, one week, one month, or one year. It is to be understood that, for any particular subject, specific dosage regimes should be adjusted over time according to the individual need and the professional judgment of the person administering or supervising the administration of a pharmaceutical composition that includes a compound of the invention

The present invention provides compositions of carbohydrate based TLR antagonists which may be prepared by conventional methods using one or more pharmaceutically acceptable excipients or adjuvants which may comprise inert diluents, sterile aqueous media and/or various non toxic solvents. The pharmaceutically acceptable carrier or diluents may be used as described in literature such as Remington: The Science and Practice of Pharmacy (20th ed.), ed. A. R. Gennaro, Lippincott Williams & Wilkins, 2000, Philadelphia, and Encyclopedia of Pharmaceutical Technology, eds. J. Swarbrick and J. C. Boylan, 1988 1999, Marcel Dekker, New York

The compositions may be presented in the form of tablets, pills, granules, powders, aqueous solutions or suspensions, injectable solutions, elixirs, or syrups, and the compositions may optionally contain one or more agents chosen from the group comprising sweeteners, flavorings, colorings, and stabilizers in order to obtain pharmaceutically acceptable preparations.

The choice of vehicle and the content of active substance in the vehicle are generally determined in accordance with the solubility and chemical properties of the product, the particular mode of administration, and the provisions to be observed in pharmaceutical practice. For example, excipients such as lactose, sodium citrate, calcium carbonate, and dicalcium phosphate and disintegrating agents such as starch, alginic acids, and certain complex silicates combined with lubricants (e.g., magnesium stearate, sodium lauryl sulfate, and talc) may be used for preparing tablets. To prepare a capsule, it is advantageous to use lactose and high molecular weight polyethylene glycols. When aqueous suspensions are used, they may contain emulsifying agents that facilitate suspension. Diluents such as sucrose, ethanol, polyethylene glycol, propylene glycol, glycerol, chloroform, or mixtures thereof may also be used.

For parenteral administration, emulsions, suspensions, or solutions of the compositions of the invention in vegetable oil (e.g., sesame oil, groundnut oil, or olive oil), aqueous-organic solutions (e.g., water and propylene glycol), injectable organic esters (e.g., ethyl oleate), or sterile aqueous solutions of the pharmaceutically acceptable salts are used. The solutions of the salts of the compositions of the invention are especially useful for administration by intramuscular or subcutaneous injection. Aqueous solutions that include solutions of the salts in pure distilled water may be used for intravenous administration with the proviso that (i) their pH is adjusted suitably, (ii) they are appropriately buffered and rendered isotonic with a sufficient quantity of glucose or sodium chloride, and (iii) they are sterilized by heating, irradiation, or microfiltration. Suitable compositions containing a compound of the invention may be dissolved or suspended in a suitable carrier for use in a nebulizer or a suspension or solution aerosol, or may be absorbed or adsorbed onto a suitable solid carrier for use in a dry powder inhaler. Solid compositions for rectal administration include suppositories formulated in accordance with known methods and containing at least one compound of formula I or II.

Dosage formulations of a compound of the invention to be used for therapeutic administration must be sterile. Sterility is readily accomplished by filtration through sterile membranes (e.g., 0.2 micron membranes) or by other conventional methods. Formulations typically are stored in lyophilized form or as an aqueous solution. The pH of the compositions of this invention is typically between 3 and 11, more desirably between 5 and 9, and most desirably between 7 and 8, inclusive. While a desirable route of administration is by injection such as intravenously (bolus and/or infusion), other methods of administration may be used. For example, compositions may be administered subcutaneously, intramuscularly, colonically, rectally, nasally, or intraperitoneally in a variety of dosage forms such as suppositories, implanted pellets or small cylinders, aerosols, oral dosage formulations, and topical formulations such as ointments, drops, and dermal patches. A compound of the invention is desirably incorporated into shaped articles such as implants, including but not limited to valves, stents, tubing, and prostheses, which may employ inert materials such as synthetic polymers or silicones, (e.g., Silastic, silicone rubber, or other commercially available polymers). Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, a TLR2 inhibitor of the invention may be coupled to a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates, and cross linked or amphipathic block copolymers of hydrogels.

A compound of the invention may also be administered in the form of liposome delivery systems, such as small unilamellar vesicles, large unilamellar vesicles, and multilamellar vesicles. Liposomes can be formed from a variety of lipids, such as cholesterol, stearylamine, or phosphatidylcholines. A compound of the invention may also be delivered using antibodies, antibody fragments, growth factors, hormones, or other targeting moieties to which the compound molecules are coupled (e.g., see Remington: The Science and Practice of Pharmacy, vide supra), including in vivo conjugation to blood components of a compound of the formula I or II, as described herein.

### In Vitro Application in Identification of TLR Antagonists:

Pharmaceutical agents that can be used in the therapeutic methods of the invention can be identified in screening methods. For example, cell-based screening methods can be used, in which cells expressing TLR are contacted with a candidate agent and the impact of the agent on the activation of TLR in the cells is determined. In one example of such a method, the effect of an agent on the activation of TLR by a known ligand (e.g., a lipopeptide,) is determined. Agents that are found to decrease or to block activation of the receptor by the ligand can then be considered for further analysis and/or for use as TLR inhibitors in therapeutic methods. Activation of TLR in these methods can be measured using, for example, a reporter system. For example, cells used in the screening assay can include a reporter gene that is under the control of a promoter that is inducible by a signaling pathway triggered by TLR activation.

In addition to cell-based methods, candidate agents can be tested in animal model systems. This may be desirable, for example, if an agent has been found to have antagonist activity in a cell-based assay or to bind to TLR in an in vitro assay (see below). For example, in animal studies, test agents can be administered to an animal model concurrently with a molecule known to activate TLR (e.g., lipopeptide), and the impact of the agent on a response in the animal that is normally triggered by activation of the receptor (e.g., cytokine induction) can be determined. Further, in vitro methods can be used. For example, a candidate compound can be assayed for whether it binds to TLR or a fragment of the receptor that includes at least a portion of the ligand binding site. Such assays can be carried out using, for example, columns or beads to which the receptor or fragment is bound.

In addition to the methods described above, additional TLR antagonists can be identified in methods in which candidate compounds are compared for TLR antagonist activity with any of the TLR antagonists described herein. Further, in addition to being compared for TLR antagonist activity, the candidate compounds can be compared with TLR2 antagonists with respect to specificity for TLR versus other receptors. Candidate compounds identified as having TLR antagonist activity that is, for example, similar to or greater than the activity of the antagonists described herein (and/or with similar or greater levels of specificity for TLR4 versus TLR2) in these assays can be tested further, for example, in appropriate animal model assays for any of the diseases or conditions described herein, as well as in human clinical studies.

Also included in the invention are compounds that are selective for TLR4 over TLR2, as well as compounds that are dual antagonists (i.e., antagonists of both TLR2 and TLR4). A compound that is selective for TLR4 over TLR2 is one that has, for example, an IC₅₀ value in a TLR4 antagonist assay, such as is described herein, that is less than that found in a TLR2 antagonist assay, such as is described herein. For example, the IC₅₀ in the TLR4 assay can be at least 5, 10, 25, or 50-fold less than the value for the same compound tested in the TLR2 assay. Compounds that are dual antagonists are those that have, for example, IC₅₀ values that are within a 5-fold range of one another using, e.g., the assays described herein. Thus, dual antagonists include those that have activities that are 1:5 5:1 with respect to one another (e.g., 1:4,1:3,1:2, 1:1, 2:1, 3:1, and 4:1). The invention also includes the use of TLR4 antagonists such as those described herein in the study of physiological and molecular pathways involved in or affected by TLR4 activation (or inactivation).

Agents that can be screened using the methods of the invention include, for example, compounds that are present in compound libraries (e.g., random libraries), as well as analogs of known TLR4 ligands (e.g., lipopeptides) that are modified to prevent rather than activate TLR2. Further, peptides that correspond to the binding site of TLR4 or its ligands, which can competitively inhibit ligand binding, can be tested. Further, antibodies or antibody fragments to the ligand or the ligand binding site of the receptor can be screened.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### General Procedure for Preparation of Monosaccharide and Disaccharide Building Blocks for all the Six Formulas

### 1. Synthesis of 1-O-(p-Methoxy phenyl)-2,3,4,6-tetra-O-acetyl-β-D-Glucopyranoside; I (RSCL-0367)

To a cooled solution of β-D-Glucose-pentaacetate (10 g, 25.6 mmol) and 4-methoxy phenol (4.8 g, 38.7 mmol) in dichloromethane (80 mL) was added trimethylsilyl trifluoromethanesulfonate (TMSOTf) (50 μL) over a period of 10 min. The reaction was stirred at room temperature for 4 h and diluted with dichloromethane (50 mL), washed with saturated aq. sodium bicarbonate solution (2×100 mL) and water (2×100 mL). The organic layer was dried over anhydrous sodium sulfate, concentrated and purified using silica gel column chromatography (20% ethyl acetate/hexane). The organic fractions containing desired product were concentrated and dried under high vacuum to provide product I as colourless solid (yield 11 g, 95%); R_{f} 0.51 (40% ethyl acetate/hexane); reported mp 102° C.; ¹H NMR (CDCl₃) δ 2.03, 2.04, 2.07, 2.08 (4 s, 12H), 3.77 (s, 3H), 4.16 (dd, 1H, J=12 Hz), 4.27 (dd, 1H, J=5.1 Hz), 4.93 (d, 1H, J=7.2 Hz), 6.81 (d, 1H), 6.94 (2d, 1H); MS m/z 477 (M+Na)⁺. Anal. Calculated for C₂₁H₂₆O₁₁: C, 55.50; H, 5.77; O, 38.73. Found: C, 55.22; H, 5.95; O, 38.82. (Slaghek T M, Nakahara Y, Ogawa T, Kamerling J P, Vliegenthart J F. Synthesis of hyaluronic acid-related di-, tri-, and tetra-saccharides having an N-acetylglucosamine residue at the reducing end. Carbohydr Res. 1994; 255:61-85.)

### 2. Synthesis of 1-O-(p-Methoxy phenyl)-β-D-glucopyranoside; II (RSCL-0369)

To a stirred solution of compound I (12 g, 24.2 mmol) in methanol (25 mL) was gradually added 2 mL of a 25% (w/v) sodium methoxide/methanol solution. The resulting solution was stirred at room temperature overnight. The mixture was neutralized by Amberlyst 15 ion-exchange (H⁺) resin. The neutralized reaction mixture was filtered, concentrated and purified using silica gel column chromatography (20% methanol/dichloromethane). The organic fractions containing desired product were concentrated and dried under high vacuum (2 mm/Hg) to provide viscous compound II (yield 6.5 g, 94%); R_{f} 0.55 (10% methanol/dichloromethane); ¹H NMR (CD₃OD) δ 3.77 (s, 3H), 4.06 (dd, 1H, J=12 Hz), 4.27 (dd, 1H, J=5.1 Hz), 4.52 (d, 1H, J=7.2 Hz), 6.81 (d, 1H), 6.94 (2d, 1H); MS m/z 309 (M+Na)⁺. Anal. Calculated for C₁₃H₁₈O₇: C, 54.54; H, 6.34; O, 39.12. Found: C, 54.44; H, 6.42; O, 39.14. (Slaghek T M, et al. Carbohydr Res. 1994; 255:61-85.)

### 3. Synthesis of 1-O-(p-Methoxy phenyl)-4,6-O-benzylidene-β-D-glucopyranoside; III (RSCL-0370)

To Compound II (3.5 g, 12.23 mmol) in anhydrous N,N-dimethylformamide (35 mL) was added dropwise benzaldehyde dimethyl acetal (3.02 mL, 20.17 mmol) followed by addition of p-toluene sulphonic acid (0.2 g). The reaction was stirred at room temperature for 16 h. N,N-Dimethylformamide was removed under high vacuum and the residue was purified using silica gel column (10% methanol/dichloromethane). The organic fractions containing desired product were concentrated and dried under high vacuum (2 mm/Hg) to provide viscous compound III (yield 2.9 g, 85%); R_{f} 0.45 (15% acetone/dichloromethane); ¹H NMR (CDCl₃) δ 3.79 (s, 3H), 4.90 (d, 1H, J=7.7 Hz), 5.58 (s, 1H), 6.81 (d, 1H), 6.94 (2d, 1H), 7.36-7.37 (m, 2H), 7.48-7.52 (m, 3H); MS m/z 397 (M+Na)⁺. Anal. Calculated for C₂₀H₂₂O₇: C, 64.16; H, 5.92; O, 29.91. Found: C, 64.08; H, 5.90; O, 30.02 (Slaghek T M, et al. Carbohydr Res. 1994; 255:61-85.)

### 4. Synthesis of 1-O-(p-Methoxy phenyl)-2,3-di-O-acetyl-4,6-O-benzylidene-β-D-glucopyranoside; IV (RSCL-0371)

To a solution of compound III (19 g, 0.050 mol) in pyridine (25 mL) at 0° C. was added acetic anhydride (25 mL) dropwise. The reaction mixture was stirred at room temperature overnight and the reaction was quenched by adding ice. Ethyl acetate was added; organic layer was washed with 1N hydrochloric acid (2×100 mL), dried over anhydrous sodium sulfate, concentrated and purified through silica gel column chromatography (20-30% ethyl acetate/hexane). The organic fractions containing desired product were concentrated and dried under high vacuum (2 mm/Hg) to give viscous compound IV (yield 21 g, 91%) R_{f} 0.62 (40% ethyl acetate/hexane); ¹H NMR (CDCl₃) δ 2.02 (s, 3H), 2.04 (s, 3H), 3.59-3.63 (m, 1H), 3.77 (s, 3H), 4.37-4.41 (dd, 1H, J=5.2 Hz & 4.8 Hz), 5.05-5.07 (d, 1H, J=7.7 Hz), 5.22-5.26 (t, 1H, J=9.2, 8 Hz), 5.36-5.41 (t, 1H, J=9.2, 9.6 Hz), 5.53 (s, 1H), 6.82-6.84 (d, 2H, J=9.2 Hz), 6.94-6.96 (d, 2H, J=9.2 Hz), 7.35-7.37 (m, 1H), 7.43-7.47 (m, 6H); MS m/z 481 (M+Na)⁺. Anal. Calculated for C₂₄H₂₆O₉: C, 62.88; H, 5.72; O, 31.41. Found: C, 62.74; H, 5.79; O, 31.47.

### 5. Synthesis of 1-O-(p-Methoxy phenyl)-2,3-di-O-acetyl-β-D-glucopyranoside; V (RSCL-0372)

To compound IV (31.5 g, 0.068 mol) in dichloromethane (250 mL) and water (6.5 mL) was added trifluoroacetic acid (56 mL) dropwise with vigorous stirring at room temperature. The reaction mixture was stirred for 3.5 hr, diluted with dichloromethane (200 mL), washed with 10% sodium bicarbonate solution (2×100 mL) and water (2×100 mL), dried over anhydrous sodium sulfate and concentrated. The residue thus obtained was purified by column chromatography (1-3% methanol/dichloromethane). The organic fractions containing desired product were concentrated and dried under high vacuum (2 mm/Hg) to give viscous product V (yield 20 g, 80%) R_{f} 0.26 (15% acetone/dichloromethane); ¹H NMR (CDCl₃) δ 2.07 (s, 3H), 2.11 (s, 3H), 3.51-3.55 (m, 1H), 3.77 (s, 3H), 3.82-3.88 (m, 2H), 3.93-3.97 (dd, 1H, J=2.8 Hz & 3.2 Hz), 4.99-5.01 (d, 1H, J=7.3 Hz), 5.08-5.18 (m, 2H), 6.81-6.83 (d, 2H, J=8.8 Hz), 6.91-6.93 (d, 2H, J=8.8 Hz); MS m/z 393 (M+Na)⁺. Anal. Calculated for C₁₇H₂₂O₉: C, 55.13; H, 5.99; O, 38.88. Found: C, 55.33; H, 5.87; O, 38.80.

### 6. Synthesis of 1-O-(p-Methoxy phenyl)-2,3-di-O-acetyl-6-O-levulinoyl-β-D-glucopyranoside; VI (RSCL-0373)

To a solution of compound V (15 g, 0.04 mol) in dichloromethane (150 mL) was added levulinic acid (8.2 mL, 0.08 mol) and 2-chloro-1-methyl-pyridinium iodide (26.5 g, 0.10 mol). The reaction mixture was stirred at room temperature for 15 min. To this was added 1,4-diazabicyclo [2.2.2] octane (17.5 g, 0.15 mol) and the reaction was stirred for an hour. Reaction mixture was filtered through celite, diluted with dichloromethane (200 mL) and washed with saturated sodium bicarbonate solution (4×150 mL), dried over anhydrous sodium sulfate, concentrated and column purified (1-3% methanol/dichloromethane). The organic fractions containing desired product were concentrated and dried under high vacuum (2 mm/Hg) to give viscous product VI (yield 7.4 g, 90%) R_{f} 0.67 (15% acetone/dichloromethane); ¹H NMR (CDCl₃) δ 2.08 (s, 3H), 2.12 (s, 3H), 2.19 (s, 3H), 2.60-2.63 (m, 2H), 2.77-2.80 (m, 2H), 3.55 (s, 2H), 3.60-3.65 (m, 1H), 3.77 (s, 3H), 4.30-4.34 (dd, 1H, J=2.4 Hz, 2.0 Hz), 4.55-4.59 (dd, 1H, J=4 Hz, 4.4 Hz), 4.93-4.95 (d, 1H, J=7.2 Hz), 5.09-5.20 (m, 2H), 6.80-6.83 (d, 2H, J=9.2 Hz), 6.93-6.96 (d, 2H, J=8.8 Hz); MS m/z 491 (M+Na)⁺. Anal. Calculated for C₂₂H₂₈O₁₁: C, 56.41; H, 6.02; O, 37.57. Found: C, 56.40; H, 5.99; O, 37.61.

### 7. Synthesis of 1-O-(p-Methoxy phenyl)-2,3-di-O-acetyl-4-O-chloroacetyl-6-O-levulinoyl-β-D-glucopyranoside; VII (RSCL-0393)

To a stirred solution of compound VI (2.4 g, 5.12 mmol) in dry dichloromethane (20 mL) and dry pyridine (3.8 mL) was added chloroacetic anhydride (1.31 g, 7.69 mmol) at 0° C. The reaction mixture was stirred at this temperature for 0.5 h and then quenched with ice. The reaction was diluted with dichloromethane (50 mL), washed with saturated sodium bicarbonate solution (20 mL) followed by water (20 mL), dried over anhydrous sodium sulfate, concentrated, purified using silica gel column chromatography (1-2% acetone/dichloromethane). The organic fractions containing desired product were concentrated and dried under high vacuum (2 mm/Hg) to give viscous product VII (yield 2.1 g, 78%) R_{f} 0.74 (10% acetone/dichloromethane); ¹H NMR (CDCl₃) δ 2.08 (s, 3H), 2.12 (s, 3H), 2.19 (s, 3H), 2.60-2.63 (m, 2H), 2.77-2.80 (m, 2H), 3.55 (s, 2H), 3.60-3.65 (m, 1H), 3.77 (s, 3H), 4.30-4.34 (dd, 1H, J=2.4 Hz, 2.0 Hz), 4.55-4.59 (dd, 1H, J=4 Hz, 4.4 Hz), 4.93-4.95 (d, 1H, J=7.2 Hz), 5.09-5.20 (m, 2H), 6.80-6.83 (d, 2H, J=9.2 Hz), 6.93-6.96 (d, 2H, J=8.8 Hz), MS m/z 568 (M+Na)⁺. Anal. Calculated for C₂₄H₂₉ClO₁₂: C, 52.90; H, 5.36; Cl, 6.51; O, 35.23. Found: C, 52.74; H, 5.29; Cl, 6.48; O, 35.49.

### 8. Synthesis of 2,3-Di-O-acetyl-4-O-chloroacetyl-6-O-levulinoyl-β-D-glucopyranoside; VIII (RSCL-0395)

To a stirred solution of compound VII (2.1 g, 3.85 mmol) in toluene: acetonitrile: water (1:1:1, 312 mL) was added ceric ammonium nitrate (21 g, 38.56 mmol). The reaction mixture was stirred at room temperature for 1 hr and diluted with ethyl acetate. Organic layer was washed with sodium bicarbonate solution (40 mL) followed by water (50 mL), dried over anhydrous sodium sulfate, concentrated and purified on silica gel column (5-10% acetone/dichloromethane). The organic fractions containing desired product were concentrated and dried under high vacuum (2 mm/1 g) to furnish viscous compound VIII (yield 1.2 g, 70%) R_{f} 0.25 (10% acetone/dichloromethane); ¹H NMR (CDCl₃) δ 2.08 (s, 3H), 2.12 (s, 3H), 2.19 (s, 3H), 2.60-2.63 (m, 2H), 2.77-2.80 (m, 2H), 3.55 (s, 2H), 3.60-3.65 (m, 1H), 3.77 (s, 3H), 4.30-4.34 (dd, 1H, J=2.4 Hz, 2.0 Hz), 4.55-4.59 (dd, 1H, J=4 Hz, 4.4 Hz), 4.93-4.95 (d, 1H, J=7.2 Hz), 5.09-5.20 (m, 2H), MS m/z 461 (M+Na)⁺. Anal. Calculated for C₁₇H₂₃ClO₁₁: C, 46.53; H, 5.28; Cl, 8.08; O, 40.11. Found: C, 46.50; H, 5.29; Cl, 8.06; O, 40.15.

### 9. Synthesis of 2,3-Di-O-acetyl-4-O-chloroacetyl-6-O-levulinoyl-α-D-glucopyranosyl trichloroacetimidate; IX (RSCL-0408)

To a solution of compound VIII (1.2 g, 2.73 mmol) in dry dichloromethane (14 mL) and trichloroacetonitrile (2.6 mL, 27.3 mmol) was added 1,8-diazabicyclo [5.4.0] undec-7-ene (103 μL, 0.68 mmol). The reaction was stirred at room temperature for 3 hr, concentrated and the residue purified by silica gel column chromatography (1-5% acetone/dichloromethane). The organic fractions containing desired product were concentrated and dried under high vacuum to yield compound IX as syrup (yield 1.27 g, 80%) R_{f} 0.37 (35% ethyl acetate/hexane); ¹H NMR (CDCl₃) δ 2.08, (s, 3H), 2.12 (s, 3H), 2.19 (s, 3H), 2.60-2.63 (m, 2H), 2.77-2.80 (m, 2H), 3.55 (s, 2H), 3.60-3.65 (m, 1H), 3.77 (s, 3H), 4.30-4.34 (dd, 1H, J=2.4 Hz, 2.0 Hz), 4.55-4.59 (dd, 1H, J=4 Hz, 4.4 Hz), 4.93-4.95 (d, 1H, J=7.2 Hz), 5.09-5.20 (m, 2H), 8.72 (s, 1H, NH).

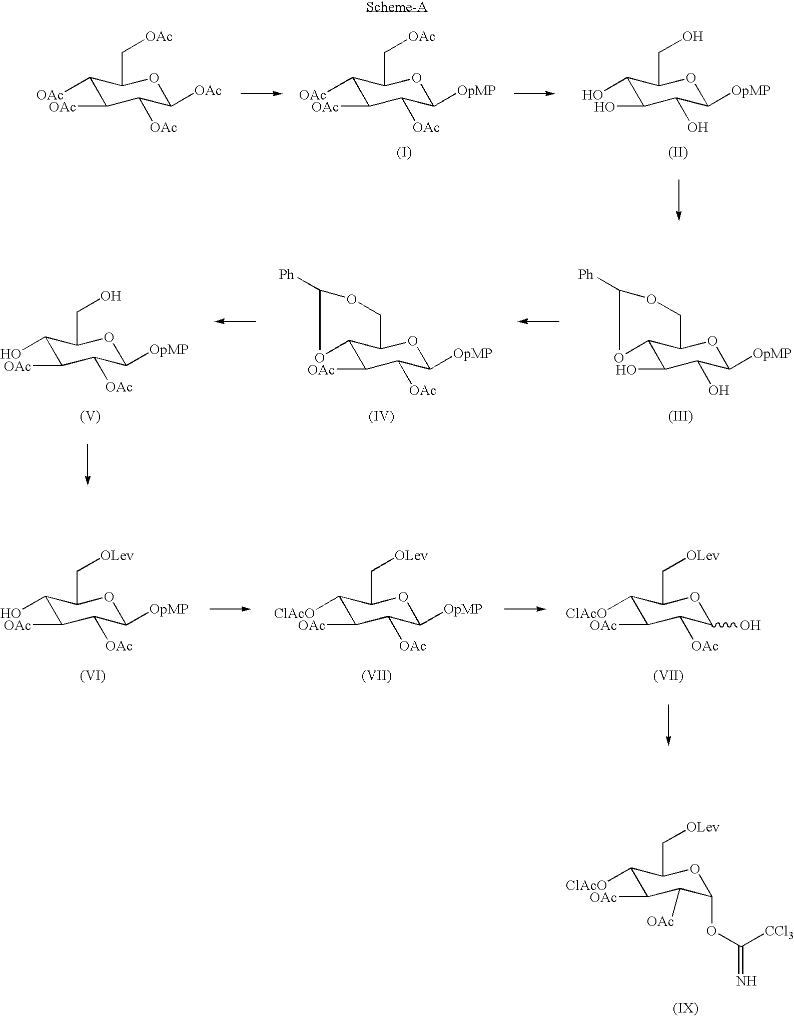

### 10. Synthesis of 1,3,4,6-Tetra-O-acetyl-2-deoxy-2-trichloroacetamido-α/β-D-glucopyranoside; X (RSCL-0397)

Trichloroacetyl chloride (8.4 mL, 75 mmol) was added dropwise at room temperature to a vigorously stirred solution of D-glucosamine hydrochloride (10.78 g, 50 mmol) and sodium bicarbonate (12.6 g, 150 mol) in water (100 mL). The mixture was stirred for 1 hr, neutralized with 1M hydrochloric acid, organic layer was separated out, concentrated, and dried in vacuo. The residue was stirred for 2 hr at 0° C. with methanol (100 mL), the salts were filtered off, and the filtrate was concentrated. Crystallization of the residue from cold water afforded 8 g of 2-deoxy-2-trichloroacetamido-D-glucopyranose, which was dissolved in pyridine (80 mL). The solution was cooled to 0° C., acetic anhydride (50 mL) was added dropwise. The reaction was stirred at room temperature overnight, filtered through celite and concentrated to give compound X (yield 9 g, 75%) as a mixture of α and β anomers in the ratio of 9:1; R_{f} 0.42 (35% Ethyl acetate/Hexane); reported mp 136° C.; ¹H NMR (CDCl₃) δ 2.04 (s, 3H), 2.10 (s, 3H), 2.18 (s, 3H), 4.04 (m, 1H), 4.08 (dd, 1H, J=2.4 Hz), 4.28 (dd, 1H, J=4.5 Hz & 13 Hz), 4.34 (m, 1H), 4.35-4.41 (m, 1H), 5.24 (t, 1H, J=9.5 Hz), 5.36 (dd, 1H, J=10.5 Hz & 9.5 Hz), 5.81 (d, 1H, J=8.5 Hz), 6.31 (d, 1H, J=3.6 Hz), 6.80 (d, 2H, J=8.5 Hz). Anal. Calculated for C₁₆H₂₀Cl₃NO₁₀: C, 39.00; H, 4.09; Cl, 21.59; N, 2.84; O, 32.47. Found: C, 39.00; H, 4.10; Cl, 21.62; N, 2.82; O, 32.46. (Blatter C, Beau J M, Jacquinet J C. The use of 2-deoxy-2-trichloroacetamido-D-glucopyranose derivatives in syntheses of oligosaccharides. Carbohydr Res. 1994; 260: 189-202.)

### 11. Synthesis of 1-O-(p-Methoxy phenyl)-2-deoxy-2-trichloroacetamido-3,4,6-tri-O-acetyl-α-D-glucopyranoside; XI (RSCL-0398)

To a stirred solution of 1,3,4,6-Tetra-O-acetyl-2-deoxy-2-trichloroacetamido-α/β-D-glucopyranoside X (10 g, 20.32 mmol) and p-methoxy phenol (7.56 g, 60.97 mmol) in dry dichloromethane at 0° C. was added TMSOTf (4.4 mL, 1.2 eq) over a period of 20 minutes. The reaction was stirred at room temperature overnight, diluted with dichloromethane. Organic layer was washed with saturated sodium bicarbonate solution (2×200 mL) followed by water (2×200 mL), dried over anhydrous sodium sulfate, concentrated and purified using silica gel column chromatography (30% ethyl acetate/hexane). The organic fractions containing desired product were concentrated and dried under high vacuum to give compound XI (yield 9.1 g, 80%) R_{f} 0.47 (35% ethyl acetate/hexane); mp 112° C.; ¹H NMR (CDCl₃) δ 2.06 (s, 6H), 2.08 (s, 1H), 3.78 (s, 3H), 4.09-4.1 (d, 1H, J=2.4 Hz), 4.13-4.12 (d, 1H, J=2.4 Hz), 4.17-4.21 (m, 1H), 4.26-4.30 (dd, 1H, J=4 Hz, 4.4 Hz), 4.35-4.41 (m, 1H), 6.83-6.85 (d, 2H, J=9.2 Hz), 7.01-7.03 (d, 2H, J=9.2 Hz), 7.09-7.07 (d, NH, J=9.2 Hz); MS m/z 575 (M+NH₄)⁺. Anal. Calculated for C₂₁H₂₄Cl₃NO₁₀: C, 45.30; H, 4.34; Cl, 19.10; N, 2.52; O, 28.74. Found: C, 45.20; H, 4.37; Cl, 19.22; N, 2.50; O, 28.71.

### 12. Synthesis of 1-O-(p-Methoxy phenyl)-2-deoxy-2-trichloroacetamido-α-D-glucopyranoside; XII (RSCL-0399)

A solution of compound XI (9 g, 16.10 mmol) in dry methanol (100 mL) was treated with sodium methoxide (0.1N, 10 mL) for 4 hr at room temperature. The mixture was then neutralized with Amberlyst 15 ion-exchange resin, filtered, concentrated and purified using silica gel column chromatography (10% methanol/dichloromethane). The organic fractions containing desired product were concentrated and dried under high vacuum to give compound XII (yield 6.6 g, 95%), R_{f} 0.23 (10% methanol/dichloromethane); mp 166.3° C.; ¹H NMR (CD₃OD) δ 3.74 (s, 3H), 3.75-3.84 (m, 4H), 3.96-3.95 (d, 1H, J=3.6 Hz), 3.99-3.98 (d, 1H, J=3.2 Hz), 4.02-4.07 (m, 1H), 5.38-5.39 (d, 1H, J=3.2 Hz), 6.82-6.84 (d, 2H, J=9.2 Hz), 7.05-7.07 (d, 2H, J=9.2 Hz); MS m/z 454 (M+Na)⁺. Anal. Calculated for C₁₅H₁₈Cl₃NO₇: C, 41.83; H, 4.21; Cl, 24.70; N, 3.25; O, 26.01. Found: C, 41.81; H, 4.13; Cl, 24.66; N, 3.24; O, 26.16.

### 13. Synthesis of 1-O-(p-Methoxy phenyl)-2-deoxy-2-trichloroacetamido-4,6-O-benzylidene-α-D-glucopyranoside; XIII (RSCL-0400)

To a solution of compound XII (5 g, 11.62 mmol) in dry N,N-dimethylformamide (15 mL) and dry tetrahydrofuran (47 mL) was added benzaldehyde dimethyl acetal (3.5 mL, 23.25 mmol) and p-toluene sulphonic acid (0.3 g). The reaction mixture was stirred at room temperature overnight. The mixture was neutralized with triethylamine, concentrated and purified using silica gel column chromatography (30% ethyl acetate/hexane). The organic fractions containing desired product were concentrated and dried under high vacuum to give compound XIII (yield 5.4 g, 90%) R_{f} 0.64 (40% ethyl acetate/hexane); mp 161.5° C.; ¹H NMR (CDCl₃) δ 3.67-3.72 (t, 1H, J=9.2 Hz), 3.78 (s, 1H), 4.02-4.09 (m, 1H), 4.22-4.34 (m, 2H), 5.50-5.49 (d, 1H, J=3.2 Hz), 5.59 (s, 1H), 6.83-6.85 (d, 2H, J=9.2 Hz), 6.98-7.00 (d, 2H, J=9.2 Hz), 7.04-7.06 (d, NH, J=8.4 Hz), 7.39-7.66 (m, 5H); MS m/z 519 (M+H)⁺. Anal. Calculated for C₂₂H₂₂Cl₃NO₇: C, 50.93; H, 4.27; Cl, 20.50; N, 2.70; O, 21.59. Found: C, 50.90; H, 4.22; Cl, 20.48; N, 2.73; O, 21.67.

### 14. Synthesis of p-Methoxy phenyl-O-(2,3-di-O-acetyl-4-O-chloroacetyl-6-O-levulinoyl-β-D-glucopyranosyl)-(1-3)-2-deoxy-2-trichloroacetamido-4,6-O-benzylidene-α-D-glucopyranoside; XIV (RSCL-0409)

Compound XIII (600 mg, 1.15 mmol) and IX (1.17 g, 2.02 mmol, 1.75 eq,) were dissolved in anhydrous dichloromethane (14 mL). Activated powdered molecular sieves were added and the reaction mixture was stirred at room temperature for 15 min. The temperature was lowered to 0° C., TMSOTf solution (10.5 μL, 0.057 mmol) was added dropwise. The reaction mixture was stirred at 0° C. for 30 min after which TLC showed complete disappearance of compound IX. The reaction was quenched with triethylamine and filtered through celite and the filtrate was concentrated. The residue was purified using silica gel column (40% ethyl acetate/hexane). The organic fractions containing desired product were concentrated and dried under high vacuum to give disaccharide XIV (Yield 975 mg, 91%) R_{f} 0.62 (50% ethyl acetate/hexane); mp 165.8° C.; ¹H NMR (CDCl₃) δ 1.97 (s, 3H), 2.03 (s, 3H), 2.14 (s, 3H), 2.40-2.48 (m, 2H), 2.55-2.65 (m, 2H), 2.74-2.82 (m, 2H), 3.63-3.68 (m, 2H), 3.76 (s, 3H), 3.82-3.84 (m, 3H), 3.95 (s, 1H), 3.97 (s, 1H), 4.00-4.14 (m, 3H), 4.19-4.18 (d, 1H, J=2.8 Hz), 4.21-4.26 (m, 2H), 4.39-4.46 (m, 3H), 4.84-4.86 (m, 1H), 5.00-5.05 (m, 2H), 5.10-5.15 (m, 1H), 5.37 (d, 1H, J=2.8 Hz), 5.60 (s, 1H), 6.80-6.83 (d, 2H, J=9.2 Hz), 6.91-6.95 (d, 2H, J=9.2 Hz), 7.34-7.37 (m, 3H), 7.50-7.53 (m, 2H); MS m/z 957 (M+NH₄)⁺. Anal. Calculated for C₃₉H₄₃Cl₄NO₁₇: C, 49.85; H, 4.61; Cl, 15.09; N, 1.49; O, 28.95. Found: C, 49.88; H, 4.60; Cl, 15.00; N, 1.44; O, 29.08.

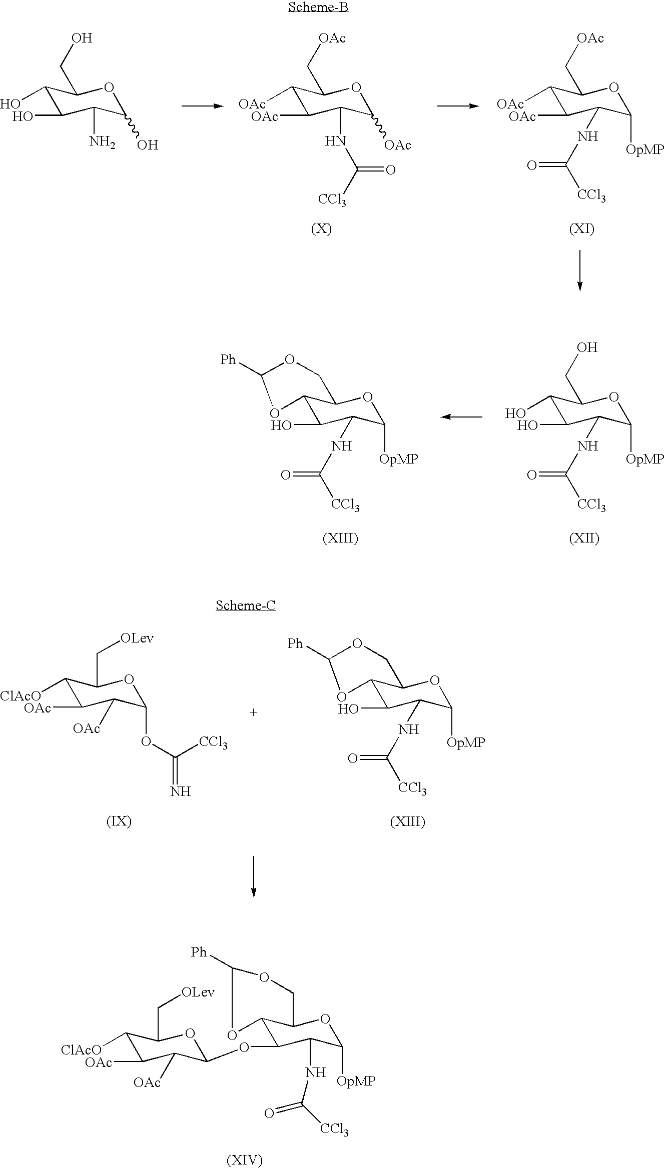

### 15. Synthesis of p-Methoxy phenyl-O-(2,3-di-O-acetyl-6-O-levulinoyl-β-D-glucopyranosyl)-(1-3)-2-deoxy-2-trichloroacetamido-4,6-O-benzylidene-α D-glucopyranoside; XV (RSCL-0440)

To a solution of compound XIV (460 mg, 0.48 mmol) in toluene:ethanol (1:1, 50 mL) was added 1,4-diazabicyclo [2.2.2] octane (17.5 g, 0.15 mol) The reaction was stirred at 55° C. for 4 h and diluted with dichloromethane (50 mL), washed with aq. 1M hydrochloric acid (50 mL) and water (2×100 mL). The organic layer was dried over anhydrous sodium sulfate, concentrated and purified using silica gel column chromatography (30% ethyl acetate/hexane). The organic fractions containing desired product were concentrated and dried under high vacuum to provide product XV (yield 323 mg, 77%); R_{f} 0.37 (60% ethyl acetate/hexane); mp 81° C.; ¹H NMR (CDCl₃) δ 1.24 (s, 6H), 1.57 (s, 4H), 2.03 (s, 3H), 2.05 (s, 3H), 2.09 (s, 1H), 2.12-2.13 (m, 1H), 2.14 (s, 2H), 2.16-2.20 (m, 1H), 2.50-2.57 (m, 2H), 2.63 (s, 1H), 2.66-2.72 (m, 2H), 2.84 (s, 1H), 3.77 (s, 3H), 3.78-3.85 (m, 2H), 4.08-4.11 (m, 1H), 4.22-4.41 (m, 5H), 4.79-4.81 (m, 1H), 4.92-4.95 (m, 2H), 5.39 (d, 1H, J=3.2 Hz), 5.60 (s, 1H), 6.81-6.83 (d, 2H, J=9.2 Hz), 6.92-6.96 (d, 2H, J=9.2 Hz), 7.34-7.37 (m, 3H), 7.50-7.53 (m, 2H); MS m/z 887 (M+Na)⁺. Anal. Calculated for C₃₇H₄₂Cl₃NO₁₆: C, 51.49; H, 4.90; Cl, 12.32; N, 1.62; O, 29.66. Found: C, 51.32; H, 4.85; Cl, 12.42; N, 1.80; O, 29.61.

### 16. Synthesis of p-Methoxy phenyl-O-(2,3-di-O-acetyl-4-O-sulfo-6-O-levulinoyl-β-D-glucopyranosyl)-(1-3)-2-deoxy-2-trichloroacetamido-4,6-O-benzylidene-α-D-glucopyranoside; XVI (RSCL-0621)

To a solution of compound XV (100 mg, 0.11 mmol) in dry N,N-dimethylformamide (8 mL) was added sulfur trioxide-trimethylamine complex (242 mg, 1.73 mmol). The reaction was stirred under argon at 50° C. for 4 hr. Methanol (3 mL) was added and reaction was stirred at room temperature for 15 min. The solvents were removed under vacuum and residue was purified using silica gel column chromatography (15% methanol/dichloromethane). The organic fractions containing desired product were concentrated and dried under high vacuum (2 mm/Hg) to provide compound XVI (yield 70 mg, 64%); R_{f} 0.14 (60% ethyl acetate/hexane); mp 142° C.; ¹H NMR (CDCl₃) δ 1.88 (s, 3H), 1.97 (s, 3H), 2.02 (s, 3H), 2.28-2.43 (m, 6H), 3.67 (s, 3H), 3.69-3.75 (m, 4H), 3.95-3.99 (m, 2H), 4.12-4.16 (m, 1H), 4.24-4.37 (m, 7H), 4.85 (d, 1H, J=7.6 Hz), 4.95-4.91 (t, 2H, J=8 Hz), 5.08-5.13 (t, 1H, J=9.6 Hz), 5.27 (d, 1H, J=3.2 Hz), 5.50 (s, 1H), 6.70-6.72 (d, 2H, J=9.2 Hz), 6.84-6.86 (d, 2H, J=8.8 Hz), 7.22-7.26 (m, 4H), 7.40-7.41 (m, 3H), 7.58-7.60 (m, 1H); MS m/z 942 (M−H)⁻. Anal. Calculated for C₃₇H₄₂Cl₃NO₁₉S: C, 47.12; H, 4.49; Cl, 11.28; N, 1.49; O, 32.23; S, 3.40. Found: C, 47.10; H, 4.40; Cl, 11.32; N, 1.56; O, 32.17; S, 3.45.

### 17. Synthesis of p-Methoxy phenyl-O-(2,3,4-tri-O-acetyl-6-O-levulinoyl-β-D-glucopyranosyl)-(1-3)-2-deoxy-2-trichloroacetamido-4,6-O-benzylidene-α-D-glucopyranoside; XVII (RSCL-0625)

To a solution of compound XV (200 mg, 0.23 mmol) in pyridine (1 mL) at 0° C. was added acetic anhydride (0.5 mL) dropwise. The reaction mixture was stirred at room temperature overnight and the reaction was quenched by adding ice. Ethyl acetate was added; organic layer was washed with 1N hydrochloric acid (2×10 mL), dried over anhydrous sodium sulfate, concentrated and purified through silica gel column chromatography (20-30% ethyl acetate/hexane). The organic fractions containing desired product were concentrated and dried under high vacuum to give compound XVII (yield 205 mg, 98%) R_{f} 0.55 (60% ethyl acetate/hexane); mp 88.8° C.; ¹H NMR (CDCl₃) δ 1.96 (s, 3H), 2.00 (s, 3H), 2.02 (s, 3H), 2.13 (s, 3H), 2.41-2.46 (m, 1H), 2.55-2.62 (m, 2H), 2.73-2.80 (m, 1H), 3.61-3.65 (m, 1H), 3.76 (s, 3H), 3.80-3.84 (m, 2H), 4.01-4.08 (m, 2H), 4.19-4.25 (m, 3H), 4.41-4.45 (m, 2H), 4.85 (d, 1H, J=8), 4.94-5.12 (m, 3H), 5.37 (d, 1H, J=3.2 Hz), 5.60 (s, 1H), 6.80-6.83 (d, 2H, J=9.2 Hz), 6.91-6.95 (d, 2H, J=9.2 Hz), 7.34-7.36 (m, 4H), 7.50-7.53 (m, 2H); MS m/z 928 (M+Na)⁺. Anal. Calculated for C₃₉H₄₄Cl₃NO₁₇: C, 51.75; H, 4.90; Cl, 11.75; N, 1.55; O, 30.05. Found: C, 51.64; H, 4.92; Cl, 11.71; N, 1.44; O, 30.29.

### 18. Synthesis of p-Methoxy phenyl-O-(2,3,4-tri-O-acetyl-β-D-glucopyranosyl)-(1-3)-2-deoxy-2-trichloroacetamido-4,6-O-benzylidene-α-D-glucopyranoside; XVIII (RSCL-0626)

To a solution of compound XVII (150 mg, 0.16 mmol) in ethanol:toluene (2:1, 9 mL) was added hydrazine acetate (76 mg, 0.82 mmol). The reaction mixture was stirred at room temperature for 1 hr, concentrated and purified through silica get column chromatography (5-10% acetone/dichloromethane). The organic fractions containing desired product were concentrated and dried under high vacuum to give compound XVIII (yield 143 mg, 95%) R_{f} 0.60 (10% acetone/dichloromethane); mp 93° C.; ¹H NMR (CDCl₃) δ 1.91 (s, 3H), 1.97 (s, 3H), 2.00 (s, 3H), 2.08 (s, 1H), 2.95 (s, 1H), 3.39-3.42 (m, 1H), 3.50-3.56 (m, 2H), 3.77 (s, 3H), 3.80-3.89 (m, 2H), 4.06-4.12 (m, 1H), 4.27-4.31 (m, 2H), 4.44-4.50 (m, 1H), 4.76 (d, 1H, J=8 Hz), 4.88-4.93 (t 1H, J=9.6 Hz), 5.02-4.97 (t, 1H, J=9.6 Hz), 5.36 (d, 1H, J=3.6 Hz), 5.58 (s 1H), 6.82-6.85 (d, 2H, J=9.2 Hz), 6.96-6.02 (m, 3H), 7.37 (s, 2H), 7.49 (s, 2H); MS m/z 830 (M+Na)⁺. Anal. Calculated for C₃₄H₃₈Cl₃NO₁₅: C, 50.60; H, 4.75; Cl, 13.18; N, 1.74; O, 29.74. Found: C, 50.44; H, 4.69; Cl, 13.50; N, 1.86; O, 29.51.

### 19. Synthesis of p-Methoxy phenyl-O-(2,3,4-tri-O-acetyl-6-O-chloroacetyl-β-D-glucopyranosyl)-(1-3)-2-deoxy-2-trichloroacetamido-4,6-O-benzylidene-α-D-glucopyranoside; XIX (RSCL-0630)

To compound XVIII (500 mg, 0.61 mmol) in dichloromethane (5 mL) and pyridine (1 mL) was added chloroacetic anhydride (160 mg) at 0° C. The reaction mixture was stirred at 0° C. for 2 hr, diluted with ethyl acetate (20 mL), washed with satd. sodium bicarbonate solution (2×20 mL) and water (2×20 mL), dried over anhydrous sodium sulfate and concentrated. The residue thus obtained was purified by column chromatography (20-30% ethyl acetate/hexane). The organic fractions containing desired product were concentrated and dried under high vacuum to give product XIX (yield 450 mg, 82%) R_{f} 0.68 (5% acetone/dichloromethane); mp 101° C.; ¹H NMR (CDCl₃) δ 1.90 (s, 3H), 1.92 (s, 3H), 1.95 (s, 3H), 3.54-3.56 (m, 1H), 3.70 (s, 3H), 3.74-3.78 (m, 2H), 3.81-3.91 (m, 2H), 4.01-4.10 (m, 2H), 4.11-4.14 (m, 1H), 4.17-4.23 (m, 2H), 4.31-4.36 (m, 1H), 4.72 (d, 1H, J=7.6 Hz), 4.92-5.06 (m, 2H), 5.32 (d, 1H, J=3.6 Hz), 5.52 (s, 1H), 6.76-6.78 (d, 2H, J=8.8 Hz), 6.86-6.88 (d, 1H, J=9.6 Hz), 6.90-6.92 (d, 1H, J=8.4 Hz), 7.27-7.30 (m, 2H), 7.40-7.42 (m, 2H); MS m/z 906 (M+Na)⁺. Anal. Calculated for C₃₆H₃₉Cl₄NO₁₆: C, 48.94; H, 4.45 Cl, 16.05; N, 1.59; O, 28.97. Found: C, 48.25; H, 4.80; Cl, 16.14; N, 1.42; O, 29.39.

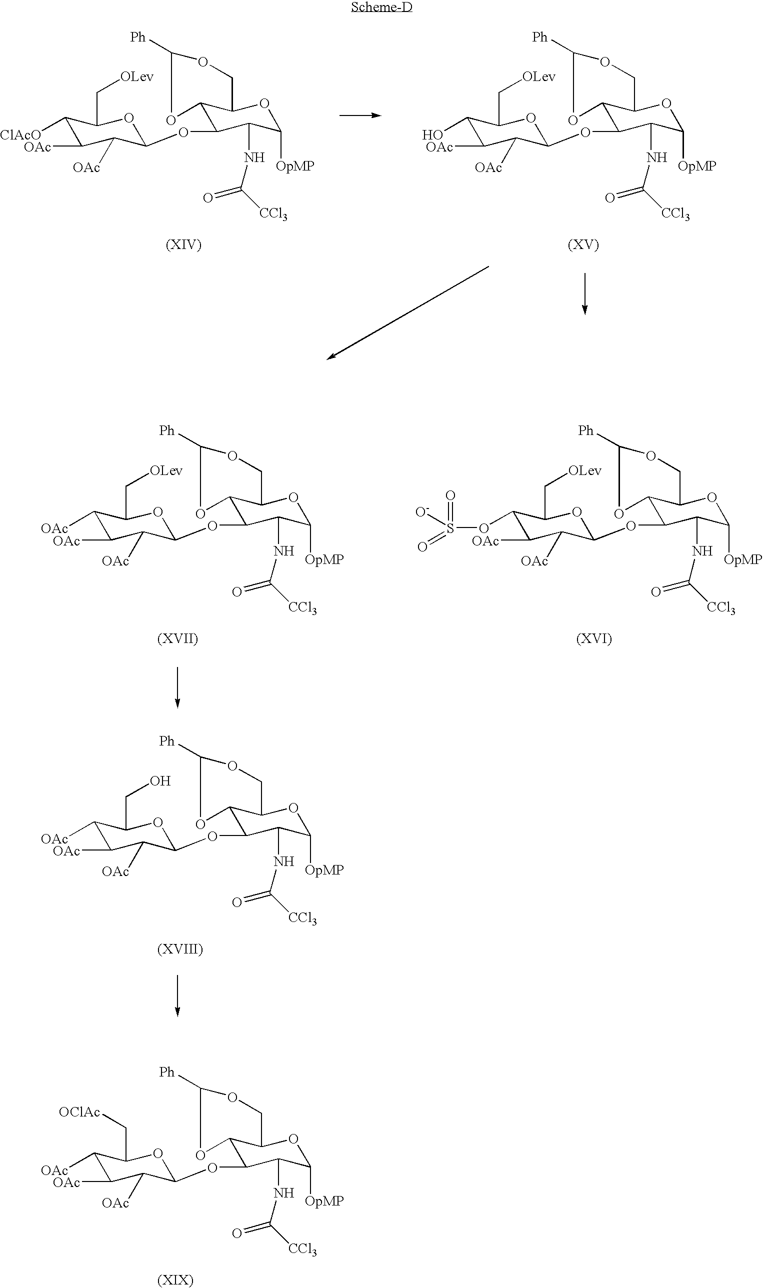

### 20. Synthesis of p-Methoxy phenyl-O-(2,3-di-O-acetyl-β-D-glucopyranosyl)-(1-3)-2-deoxy-2-trichloroacetamido-4,6-O-benzylidene-α-D-glucopyranoside; XX (RSCL-0628)

To a solution of compound XIV (100 mg, 0.10 mmol) in ethanol:toluene (2:1, 6 mL) was added hydrazine acetate (49 mg, 0.53 mmol). The reaction mixture was stirred at room temperature for 1 hr, concentrated and purified through silica gel column chromatography (40% ethyl acetate/hexane). The organic fractions containing desired product were concentrated and dried under high vacuum (2 mm/Hg) to give viscous compound XX (yield 64 mg, 79%) R_{f} 0.27 (50% ethyl acetate/hexane); ¹H NMR (CDCl₃) δ 1.88 (s, 3H), 1.99 (s, 3H), 2.01-2.12 (m, 2H), 3.31-3.32 (m, 1H), 3.51-3.63 (m, 2H), 3.70 (s, 3H), 3.73-3.81 (m, 3H), 4.02-4.05 (m, 1H), 4.20-4.25 (m, 2H), 4.38-4.43 (m, 1H), 4.68-4.69 (m, 1H), 4.83-4.87 (m, 1H), 5.29 (d, 1H, J=3.6 Hz), 5.51 (s, 1H), 6.76-6.78 (d, 2H, J=8.8 Hz), 6.89-6.92 (d, 2H, J=9.2 Hz), 7.30 (s, 2H), 7.41-7.42 (d, 2H, J=3.6 Hz); MS m/z 788 (M+Na)⁺. Anal. Calculated for C₃₂H₃₆Cl₃NO₁₄: C, 50.24; H, 4.74; Cl, 13.90; N, 1.83; O, 29.28. Found: C, 50.32; H, 4.80; Cl, 13.70; N, 1.86; O, 29.32.

### 21. Synthesis of p-Methoxy phenyl-O-(2,3-di-O-acetyl-4,6-di-O-chloroacetyl-β-D-glucopyranosyl)-(1-3)-2-deoxy-2-trichloroacetamido-4,6-O-benzylidene-α-D-glucopyranoside; XXI (RSCL-0631)

To compound XX (50 mg, 0.06 mmol) in dichloromethane (0.8 mL) and pyridine (0.4 mL) was added chloroacetic anhydride (89 mg) at 0° C. The reaction mixture was stirred at 0° C. for 3 hr, diluted with ethyl acetate (20 mL), washed with satd. sodium bicarbonate solution (2×20 mL) and water (2×20 mL), dried over anhydrous sodium sulfate and concentrated. The residue thus obtained was purified by column chromatography (20-30% ethyl acetate/hexane). The organic fractions containing desired product were concentrated and dried under high vacuum to give product XXI (yield 40 mg, 66%) R_{f} 0.68 (50% ethyl acetate/hexane); mp 90° C.; ¹H NMR (CDCl₃) δ 1.18 (s 3H), 1.47 (s, 3H), 1.90 (s, 2H), 1.95 (s, 2H), 3.56-3.61 (m, 1H), 3.71 (s, 3H), 3.73-3.77 (m, 2H), 3.81-3.93 (m, 3H), 4.00-4.07 (m, 1H), 4.11-4.14 (m, 1H), 4.17-4.23 (m, 3H), 4.31-4.37 (m, 1H), 4.73 (d, 1H, J=8 Hz), 4.90-5.09 (m, 2H), 5.32 (d, 1H, J=4 Hz), 5.51 (s, 1H), 6.76-6.78 (d, 2H, J=8.8 Hz), 6.85-6.87 (d, 1H, J=9.2 Hz), 6.90-6.92 (d, 1H, J=9.2 Hz), 7.25-7.30 (m, 3H), 7.39-7.41 (m, 2H); MS m/z 941 (M+Na)⁺. Anal. Calculated for C₃₆H₃₈Cl₅NO₁₆: C, 47.10; H, 4.17 Cl, 19.31; N, 1.53; O, 27.89. Found: C, 47.08; H, 4.20; Cl, 19.22; N, 1.52; O, 27.98.

### 22. Synthesis of p-Methoxy phenyl-O-(2,3-di-O-acetyl-4-O-chloroacetyl-β-D-glucopyranosyl)-(1-3)-2-deoxy-2-trichloroacetamido-4,6-O-benzylidene-α-D-glucopyranoside; XXII (RSCL-0629)

To a solution of compound XIV (100 mg, 0.10 mmol) in ethanol:toluene (2:1, 6 mL) was added hydrazine acetate (20 mg, 0.21 mmol). The reaction mixture was stirred at room temperature for 1 hr, concentrated and purified through silica gel column chromatography (20% ethyl acetate/hexane). The organic fractions containing desired product were concentrated and dried under high vacuum to give compound XXII (yield 70 mg, 78%) R_{f} 0.53 (50% ethyl acetate/hexane); mp 76° C.; ¹H NMR (CDCl₃) δ 1.95 (s, 3H), 1.99 (s, 3H), 3.31-3.39 (m, 1H), 3.51-3.53 (m, 1H), 3.70 (s, 3H), 3.73-3.78 (m, 2H), 3.88 (s, 2H), 4.01-4.04 (m, 1H), 4.17-4.24 (m, 3H), 4.28-4.33 (m, 2H), 4.72 (d, 1H, J=7.2 Hz), 4.83-4.88 (m, 2H), 5.33 (d, 1H, J=3.6 Hz), 5.52 (s, 1H), 6.75-6.77 (d, 2H, J=8.8 Hz), 6.86-6.92 (m, 3H), 7.28-7.31 (m, 2H), 7.40-7.42 (m, 2H); MS m/z 863.8 (M+Na)⁺. Anal. Calculated for C₃₄H₃₇Cl₄NO₁₅: C, 48.53; H, 4.43; Cl, 16.85; N, 1.66; O, 28.52. Found: C, 48.47; H, 4.41; Cl, 16.80; N, 1.76; O, 28.56.

### 23. Synthesis of 2,3-Di-O-acetyl-4-O-chloroacetyl-6-O-levulinoyl-β-D-glucopyranosyl-(1-3)-2-deoxy-2-trichloroacetamido-4,6-O-benzylidene-α/β-D-glucopyranoside; XXIII (RSCL-0627)

To a stirred solution of compound XIV (0.4 g, 0.42 mmol) in toluene: acetonitrile: water (1:1:1, 60 mL) was added ceric ammonium nitrate (2.3 g, 4.26 mmol). The reaction mixture was stirred at room temperature for 3 hr and diluted with ethyl acetate (150 mL). Organic layer was washed with sodium bicarbonate solution (100 mL) followed by water (100 mL), dried over anhydrous sodium sulfate, concentrated and purified on silica gel column (30-50% ethyl acetate/hexane). The organic fractions containing desired product were concentrated and dried under high vacuum (2 mm/Hg) to furnish viscous compound XXIII (yield 0.3 g, 84%) R_{f} 0.28 (50% ethyl acetate/hexane); ¹H NMR (CDCl₃) δ 1.96 (s, 3%), 2.02 (s, 3H), 2.17 (s, 3H), 2.42-2.47 (m, 1H), 2.53-2.67 (m, 2H), 2.73-2.84 (m, 1H), 3.61-3.65 (m, 1H), 3.71-3.84 (m, 2H), 3.93-4.10 (m, 3H), 4.14-4.31 (m, 2H), 4.79 (d, 1H, J=8 Hz), 4.98-5.04 (m, 2H), 5.08-5.13 (m, 1H), 5.27 (s, 1H), 5.57 (s, 1H), 7.30-7.36 (m, 2%), 7.50 (d, 2H, J=3.2 Hz); MS m/z 850.8 (M+NH₄)⁺. Anal. Calculated for C₃₂H₃₇Cl₄NO₁₆: C, 46.12; H, 4.47; Cl, 17.01; N, 1.68; O, 30.71. Found: C, 46.10; H, 4.44; Cl, 17.06; N, 1.70; O, 30.70.

### 24. Synthesis of 2,3-Di-O-acetyl-4-O-chloroacetyl-6-O-levulinoyl-β-D-glucopyranosyl)-(1-3)-2-deoxy-2-trichloroacetamido-α/β-D-glucopyranoside; XXIV (RSCL-0632)

To compound XXIII (0.25 g, 0.3 mmol) in dichloromethane (2 mL) and water (0.2 mL) was added trifluoroacetic acid (0.23 mL) dropwise with vigorous stirring at room temperature. The reaction mixture was stirred for 4 hr, diluted with dichloromethane (30 mL), washed with 10% sodium bicarbonate solution (2×20 mL) and water (2×20 mL), dried over anhydrous sodium sulfate and concentrated. The residue thus obtained was purified by column chromatography (4-5% methanol/dichloromethane). The organic fractions containing desired product were concentrated and dried under high vacuum (2 mm/Hg) to give viscous product XXIV (yield 0.178 g, 80%) R_{f} 0.22 (10% methanol/dichloromethane); ¹H NMR (CDCl₃) δ 1.92 (s, 3H), 2.02 (s, 3H), 2.15 (s, 3H), 2.45-2.55 (m, 2H), 2.65-2.78 (m, 2H), 3.44-3.54 (m, 1H), 3.69-3.79 (m, 2H), 3.85-3.94 (m, 2H), 4.06-4.09 (m, 2H), 4.26-4.29 (m, 1H), 4.63 (d, 1H, J=8 Hz), 4.91-5.16 (m, 2H); MS m/z 763 (M+NH₄)⁺. Anal. Calculated for C₂₅H₃₃Cl₄NO₁₆: C, 40.29; H, 4.46; Cl, 19.03; N, 1.88; O, 34.34. Found: C, 40.25; H, 4.48; Cl, 19.05; N, 1.78; O, 34.44.

### 25. Synthesis of 1-O-(p-methoxy phenyl)-2-deoxy-2-trichloroacetamido-3-O-chloroacetyl-4,6-O-benzylidene-α-D-glucopyranoside, XXV (RSCL-0477)

To a stirred solution of compound XIII (2 g, 3.86 mmol) in dry dichloromethane (20 mL) and dry pyridine (3 mL) was added chloroacetic anhydride (0.99 g, 5.79 mmol) at 0° C. followed by addition of 2,4-dimethyl amino pyridine (DMAP) (100 mg). The reaction mixture was stirred at this temperature for 0.5 hr and then quenched with ice. The reaction was diluted with dichloromethane (50 mL), washed with saturated sodium bicarbonate solution (20 mL) followed by water (20 mL), dried over anhydrous sodium sulfate, concentrated, purified using silica gel column chromatography (10-15% ethyl acetate/hexane). The organic fractions containing desired product were concentrated and dried under high vacuum to provide product XXV as colourless solid (yield 2.1 g, 91%) R_{f} 0.67 (40% ethyl acetate/hexane); mp 163° C.; ¹H NMR (CDCl₃) δ 3.78 (s, 3H), 3.82-3.91 (m, 2H), 4.09 (d, 2H, J=2.8 Hz), 4.13-4.17 (m, 1H), 4.28-4.32 (m, 1H), 4.42-4.46 (m, 1H), 5.48 (d, 1H, J=3.2 Hz), 5.56 (s, 1H), 5.65-5.70 (t, 1H, J=10 Hz), 6.86 (d, 2H, J=8.8 Hz), 7.01 (d, 2H, J=8.8 Hz), 7.15 (d, 1H, J=8.8 Hz), 7.37-7.45 (m, 5H); MS m/z 619 (M+Na)⁺. Anal. Calculated for C₂₄H₂₃Cl₄NO₈: C, 48.43; H, 3.89; Cl, 23.82; N, 2.35; O, 21.50. Found: C, 48.47; H, 3.90; Cl, 23.87; N, 2.43; O, 21.33.

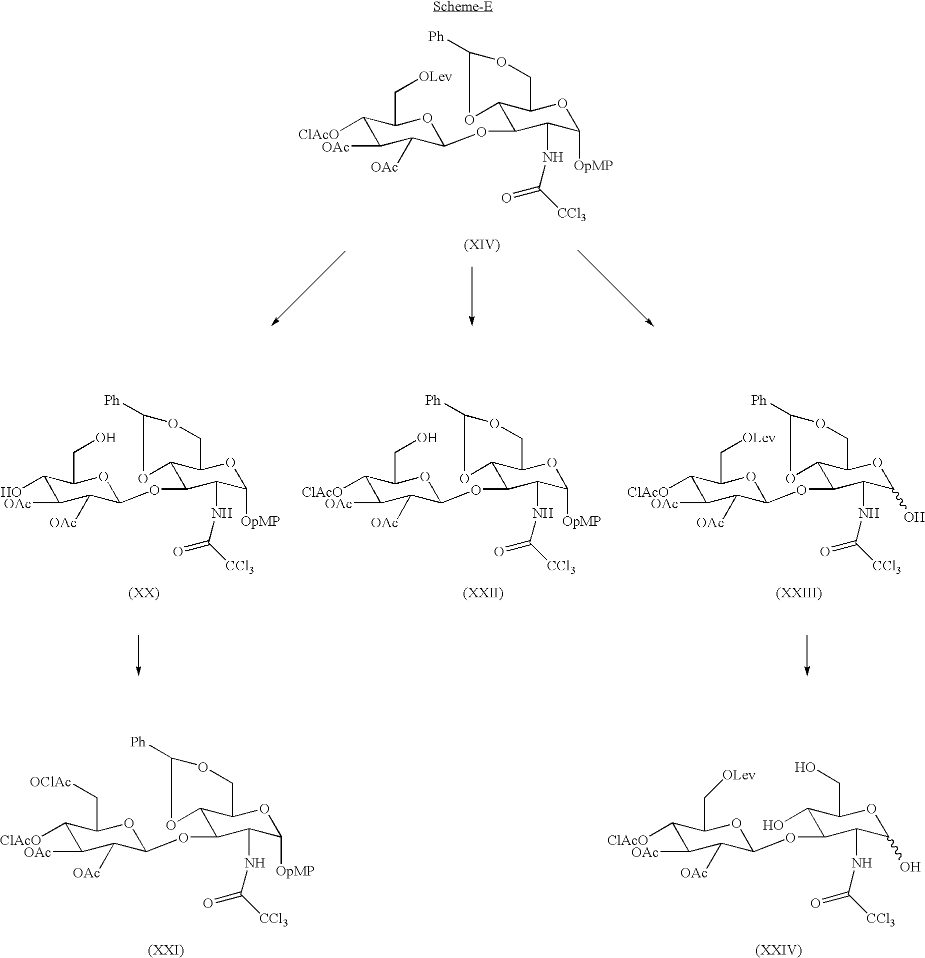

### 26. Synthesis of 2-Deoxy-2-trichloroacetamido-3-O-chloroacetyl-4,6-O-benzylidene-α-D-glucopyranoside; XXVI (RSCL-0478)

To a stirred solution of compound XXV (2.3 g, 3.86 mmol) in toluene: acetonitrile:water (1:1:1, 330 mL) was added ceric ammonium nitrate (21 g, 38.56 mmol). The reaction mixture was stirred at room temperature for 1.5 hr and diluted with ethyl acetate. Organic layer was washed with sodium bicarbonate solution (40 mL) followed by water (50 mL), dried over anhydrous sodium sulfate, concentrated and purified on silica gel column (20-40% ethyl acetate/hexane). The organic fractions containing desired product were concentrated and dried under high vacuum to furnish compound XXVI (yield 1.02 g, 54%) R_{f} 0.38 (35% Ethyl acetate/Hexane); mp 89° C.; ¹H NMR (CDCl₃) δ 3.80-3.83 (m, 3H), 3.94 (s, 1H), 4.06-4.11 (m, 4H), 4.13-4.17 (m, 1H), 4.29-4.31 (m, 2H), 5.37 (d, 1H, J=3.2 Hz), 5.54 (s, 1H), 7.37-7.44 (m, 5H); MS m/z 512 (M+Na)⁺. Anal. Calculated for C₁₇H₁₇Cl₄NO₇: C, 41.74; H, 3.50; Cl, 28.99; N, 2.86; O, 22.90. Found: C, 41.75; H, 3.55; Cl, 29.03; N, 2.82; O, 22.85.

### 27. Synthesis of 2-Deoxy-2-trichloroacetamido-3-O-chloroacetyl-4,6-O-benzylidene-α-D-glucopyranosyl trichloroacetimidate; XXVII (RSCL-0479)

To a solution of compound XXVI (1 g, 2.03 mmol) in dry dichloromethane (12 mL) and trichloroacetonitrile (2.0 mL, 20.32 mmol) was added 1,8-diazabicyclo [5.4.0] undec-7-ene (76 μL, 0.50 mmol). The reaction was stirred at room temperature for 3 hr, concentrated and the residue purified by silica gel column chromatography (10-12% ethyl acetate/hexane). The organic fractions containing desired product were concentrated and dried under high vacuum to yield compound XXVII as syrup (yield 1.1 g, 84%) R_{f} 0.55 (30% ethyl acetate/hexane); ¹H NMR (CDCl₃) δ 2.00 (s, 1H), 3.81-3.86 (t, 1H, J=10 Hz), 3.91-3.96 (t, 1H, 9.6 Hz), 4.07-4.15 (m, 4H), 4.36-4.40 (m, 1H), 4.50-4.56 (m, 1H), 5.56-5.62 (m, 3H), 6.46 (d, 1H, J=3.6 Hz), 7.02 (d, 1H, J=8.8 Hz), 7.35-7.39 (m, 3H), 7.43-7.46 (m, 2H), 8.83 (s, NH).

### 28. Synthesis of p-Methoxy phenyl-2-deoxy-2-trichloroacetamido-4,6-O-benzylidene-β-D-glucopyranosyl)-(1-4)-O-(2,3-di-O-acetyl-6-O-levulinoyl-β-D-glucopyranoside; XXVIII (RSCL-0635)

Compound VI (0.1 g, 0.21 mmol) and XXVII (0.3 g, 0.47 mmol, 2.2 eq,) were dissolved in anhydrous dichloromethane (2.5 mL). Activated powdered molecular sieves were added and the reaction mixture was stirred under argon at room temperature for 15 min. The temperature was lowered to 0° C., Boron trifluoride etherate (14.8 μL, 0.11 mmol) was added dropwise. The reaction mixture was stirred at 0° C. for 1 hr after which TLC showed complete disappearance of compound XXVII. The reaction was neutralized with triethylamine and diluted with dichloromethane (20 mL). The organic layer was washed with brine (2×10 mL), dried over anhydrous sodium sulfate, concentrated and purified using silica gel column (20-40% ethyl acetate/hexane). The organic fractions containing desired product were concentrated and dried under high vacuum to give disaccharide XXVIII (RSCL-0635) (Yield 110 mg, 55%) R_{f} 0.62 (20% acetone/toluene); mp 205° C.; ¹H NMR (CDCl₃) δ 2.06 (s, 3H), 2.07 (s, 3H), 2.17 (s, 1H), 2.19 (s, 1H), 2.36 (s, 2H), 2.36-2.42 (m, 2H), 2.58-2.63 (m, 2H), 2.71-2.84 (m, 2H), 3.21 (s, 1H), 3.36-3.39 (t, 1H, J=6.8 Hz), 3.62-3.66 (dd, 1H, J=4.4 Hz, 4.8 Hz), 3.76 (s, 3H), 4.05 (d, 1H, J=5.6 Hz), 4.09 (d, 1H, J=3.6 Hz), 4.19-4.28 (m, 1H), 4.43-4.47 (q, 1H), 4.51-4.54 (m, 1H), 4.95 (d, 1H, J=7.6 Hz), 5.05 (d, 1H, J=8.4 Hz), 5.17-5.29 (m, 2%), 5.51 (s, 1H), 5.54-5.59 (t, 1H, J=10 Hz), 6.79-6.81 (d, 2H, J=9.2 Hz), 6.89-6.92 (d, 2H, J=9.2 Hz), 7.34-7.35 (m, 2H), 7.40-7.42 (m, 2H), 7.76 (d, 1H, 10 Hz); MS m/z 957 (M+NH₄)⁺. Anal. Calculated for C₃₉H₄₃Cl₄NO₁₇: C, 49.85; H, 4.61; Cl, 15.09; N, 1.49; O, 28.95. Found: C, 49.68; H, 4.62; Cl, 15.11; N, 1.44; O, 29.15.

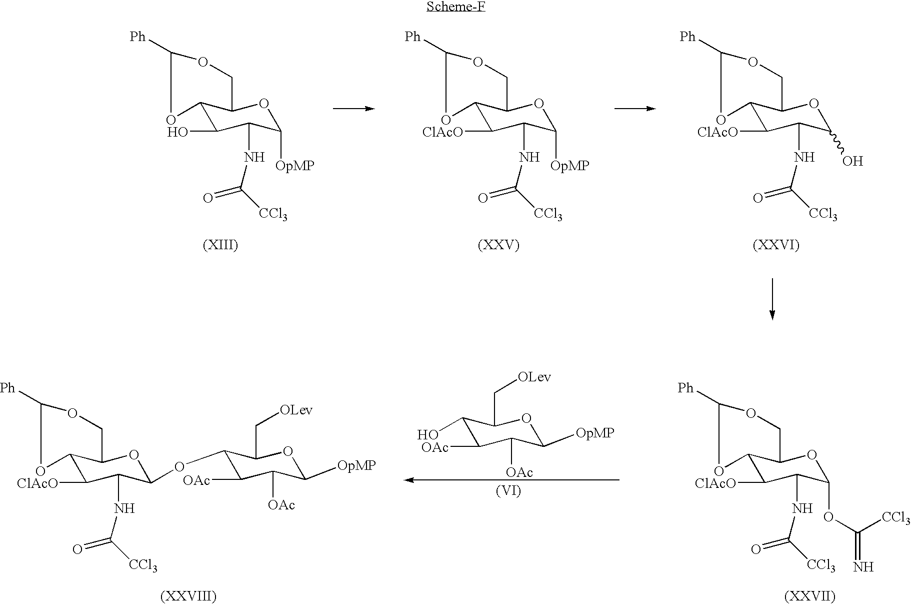

### Example 2

### Testing In Vitro of Synthesized Molecules

### Experiment 1: Comparative Activity Chart of TNF Inhibition of the Synthesized Molecules:

All the molecules synthesized as described earlier were initially pre-screened for TNF inhibition activity to identify the most effective anti-inflammatory molecule as shown in Table 1. Based on the activity as well as cell viability, RSCL-0409-highlighted in Table 1 showed the best TNF inhibiting activity, which was selected to for further in-vitro studies.

TABLE 1
Comparative analysis of anti-TNF activity of synthesized molecules

| | % TNF Inhibition | | | | % Viability | | | |
|---|---|---|---|---|---|---|---|---|
| | 100 uM | 50 uM | 10 uM | 1 uM | 100 uM | 50 uM | 10 uM | 1 uM |
| RSCL-0367 | 0 | 0 | 0 | 0 | 116 | 105 | 118 | 114 |
| RSCL-0370 | 16 | 0 | 0 | 0 | 107 | 108 | 120 | 115 |
| RSCL-0393 | 76 | 18 | 13 | 2 | 86 | 81 | 85 | 84 |
| RSCL-0400 | 0 | 0 | 0 | 0 | 63 | 70 | 82 | 93 |
| RSCL-0409 | 91 | 81 | 32 | 0 | 113 | 114 | 112 | 108 |
| RSCL-0440 | 47 | 46 | 42 | 34 | 119 | 129 | 115 | 114 |
| RSCL-0621 | 66 | 49 | 38 | 36 | 96 | 98 | 95 | 90 |
| RSCL-0625 | 31 | 22 | 27 | 31 | 97 | 99 | 96 | 90 |
| RSCL-0626 | 52 | 13 | 43 | 39 | 89 | 95 | 92 | 97 |
| RSCL-0627 | 100 | 104 | 80 | 0 | 13 | 33 | 90 | 105 |
| RSCL-0628 | 100 | 102 | 92 | 0 | 8 | 10 | 60 | 89 |
| RSCL-0629 | 100 | 102 | 94 | 0 | 11 | 13 | 57 | 99 |
| RSCL-0630 | 0 | 61 | 36 | 31 | 81 | 85 | 88 | 95 |
| RSCL-0631 | 99 | 92 | 38 | 77 | 8 | 20 | 70 | 95 |
| RSCL-0632 | 30 | 0 | 0 | 0 | 84 | 94 | 113 | 115 |
| RSCL-0635 | 0 | 0 | 0 | 0 | 81 | 82 | 89 | 97 |

### Experiment 2: Effect of RSCL-0409 (Compound 14) on Various TLR Ligands

The present invention has checked the effect of various TLR ligands on THP-1 monocytes, PBMCs and their ability to activate and release TNF-α. For this purpose about 9 TLR ligands were used. These ligands were obtained from Apotech Cat; APO-54N-018-K100 and assayed for TNF-α release in culture supernatants.

THP-1 and PBMCs (2×10⁵ cells/well) were plated in 96-well plate. The cells were pretreated with RSCL-0409 in DMSO 1 hr prior to TLR ligand treatment. Following 1 hr pre-treatment, the cells were treated with TLR ligands [TLR2, TLR5 and TLR6-75 ng/ml each, TLR3-75 μg/ml, TLR4-750 ng/ml, TLR7, TLR8 and TLR9-7.5 μg/ml) for 24 hrs. The culture supernatant were collected after the stipulated time and assayed for TNF-α release using a Duoset Enzyme-Linked ImmunoSorbent Assay (ELISA) detection Kit (R&D systems, MN 55413, USA; Cat: DY-210 E). Simultaneously supernatants were collected from cells treated with ligands without pre-treatment with RSCL-0409 and only RSCL-0409 without ligand treatment.

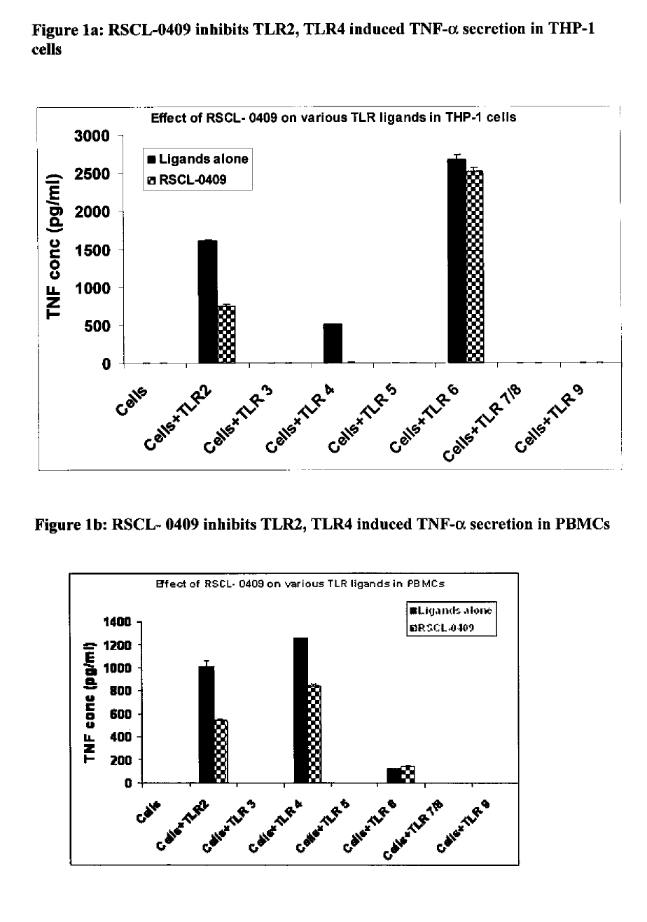

RSCL-0409 selectivity to suppress TLR2 and TLR4 mediated TNF-α production in THP-1 stimulated cells and PBMCs (FIGS. 1a and 1b). Treatment with RSCL-0409 inhibited the TNF-α secretion in TLR2 treated cells (˜54% in THP-1 cells, ˜46% in PBMC) and TLR4 (˜100% in THP-1 cells, ˜34% in PBMCs). However, there was no effect observed in cells treated with TLR6 ligand, indicating that RSCL-0409 is a potential inhibitor for signaling mechanisms induced by LPS or lipoproteins which signal through the above mentioned TLRs. We wish to mention that there is documented evidence of TLRs 3, 7, 8 and 9 to predominantly secrete interferon-gamma¹ (IFN-γ). We speculate that it might be the reason for not able to have detectable levels of TNF following stimulation with these ligands in our system. TLR2 ligand from the kit is Pam3CSK, a synthetic tripalymitoyl lipopeptide which is know to potentially activate monocytes and macrophages and TLR6 is a macrophage stimulating lipopeptide-2 and this is known to activate the cells when it heterodimerizes with TLR2. (Takeda K, Takeuchi O, Akira S. Recognition of lipopeptides by Toll-like receptors. J Endotoxin Res. 2002; 8: 459-63; Sandor F, Latz E, Re F, Mandell L, Repik G, Golenbock D T, Espevik T, Kurt-Jones E A, Finberg R W. Importance of extra- and intracellular domains of TLR1 and TLR2 in NFkappa B signaling. J Cell Biol. 2003; 162:1099-110.) It is plausible that RSCL-0409 inhibits cytokine production induced by an additive process of TLR2 and TLR4. Based on the data it is clear that RSCL-0409 suppress the activation of cells by TLRs, probably an upstream event in TLR2, 4 mediated signaling, and has the ability to recognize a lipopeptide.

### Experiment 3: Inhibition of TNF-α Secretion in THP-1 Cells by RSCL-0409 is Dose-Dependent

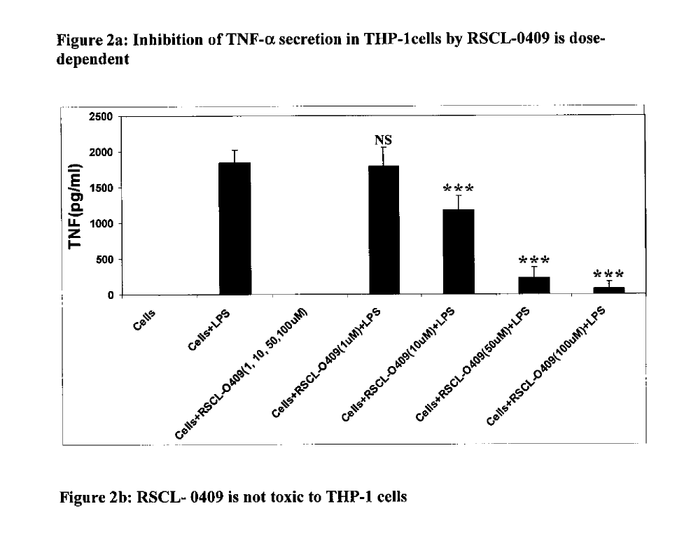

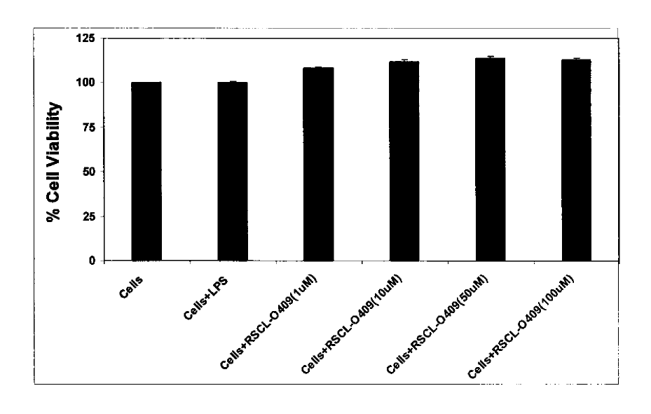

To confirm whether the inhibitory effect of RSCL-0409 is dose-dependent, the present invention has studied the ability of RSCL-0409 to inhibit TNF-α secretion from LPS (250 ng/ml) induced THP-1 monocytes (FIG. 2a).

THP-1, 2×10⁵ cells/well was plated in 96-well plate. The cells were pretreated with RSCL-0409 at various concentrations (100 μM, 50 μM, 10 μM and 1 μM) 1 hr prior to LPS stimulation. As a control group, cells were treated with LPS alone and cells treated with RSCL-0409 alone were used. TNF-α secretion was estimated in the culture supernatants following 24 hr LPS stimulation using Duoset ELISA detection Kit (R&D systems, MN, USA). *P<0.05, ***P<0.001 values are comparisons for LPS treated vs. RSCL-0409 treated, NS indicates not significant.

The toxicity of RSCL-0409 (FIG. 2b) was also by treating cells with RSCL-0409 by MTT assay. LPS induced TNF-α secretion was inhibited by RSCL-0409 in a concentration dependent manner. The viability of the cells (analyzed in tandem by MTT) was not affected by RSCL-0409 indicating its non-toxic nature.

### Experiment 4: Effect of RSCL-0409 on LPS Induced TNF-α Release in Human PBMC

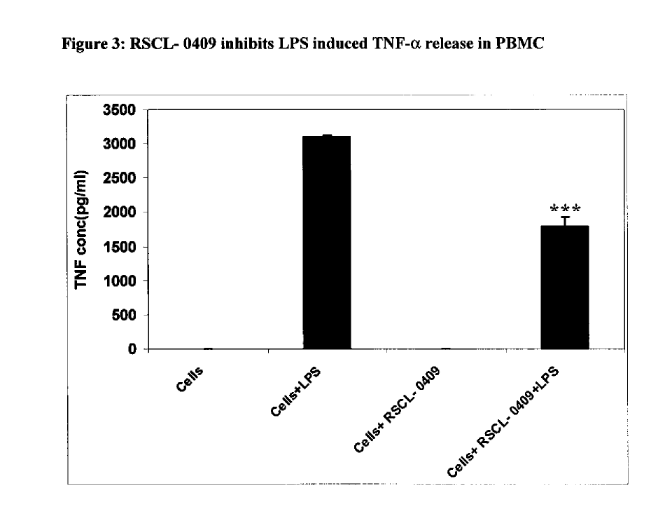

In order to evaluate the ability of RSCL-0409 to inhibit LPS induced TNF-α secretion in a physiological scenario, the present invention has tested the same in PBMC isolated from human blood (FIG. 3). We observed inhibitory effects similar to that seen in THP-1 cells. The TNF levels were not detectable in PBMCs without LPS and with RSCL-0409 treatment alone, indicating its specificity in LPS induced TNF-α through a TLR mediated process. ***P<0.001 value is for LPS treated vs. RSCL-0409 treated.

### Experiment 5: Effect of RSCL-0409 on TNF Response to Increasing Concentration of LPS

The present invention has also conducted experiments to check the ability of RSCL-0409 to inhibit LPS induced TNF-α secretion from THP-1 cells, wherein the studies involved the stimulation of THP-1 cells with increasing concentrations of LPS (62.5 ng/ml to 1000 ng/ml) with and without pre-treatment of cells with different concentrations of RSCL-0409.

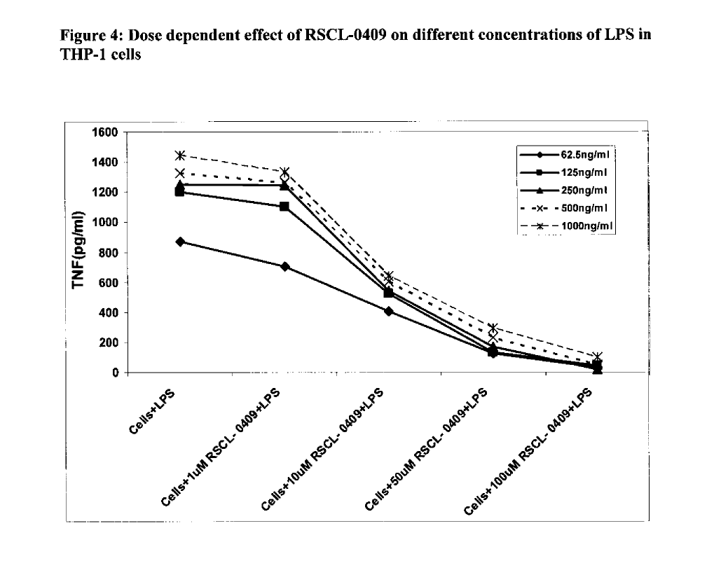

It is clearly evident (FIG. 4) that with an increased secretion of TNF-α with increasing concentration of LPS is inhibited by RSCL-0409 to varied extents on a concentration dependent manner. Further RSCL-0409 has the ability to inhibit LPS (1000 ng/ml) induced TNF-α secretion at concentration as low as 10 μM substantiating its potency as an antagonist to LPS induced stimulatory processes.

Experiment 6: Effect of RSCL-0409 on TNF-α and IL-6 mRNA Expression in THP-1 Cells in Real Time

To determine whether the suppressive effect of RSCL-0409 on the cytokine production occurs at mRNA expression level, quantitative real-time PCR was used to examine TNF-α and IL-6 mRNA expression in THP-1 cells stimulated with LPS. THP-1 cells (3×10⁶ cells/well) were stimulated with 250 ng/ml LPS in the presence or absence of RSCL-0409 at indicated concentrations for the 1 hr. Total RNA was isolated from these cells and cDNA was synthesized. LPS treated cells acted as positive control. All quantitative real-time PCR (TaqMan™) primers and probes were obtained from Applied Biosystems (ABI), Weiterstadt, Germany. For detection of TNF-α, pre-developed assay reagents (Universal master mix as obtained from Applied Biosystems included all reagents including Taq-polymerase apart from specific primers and probes) were used. The PCR was performed utilizing 1 μl cDNA per reaction in triplicates of 25 μl volume on an ABI 7500 Realtime PCR machine using a 2-step PCR protocol.

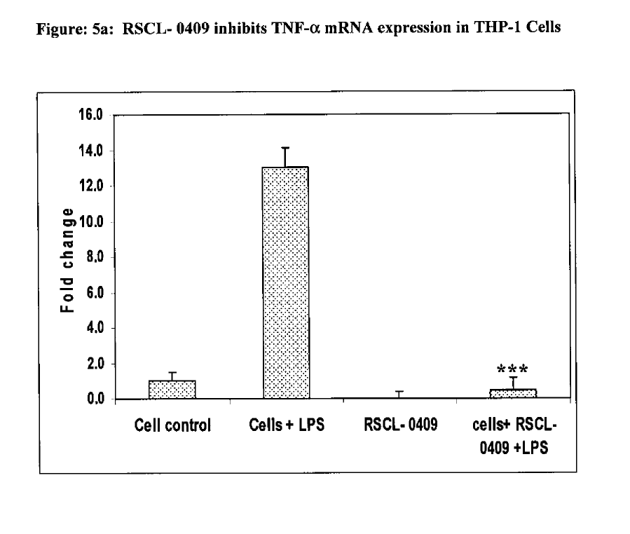

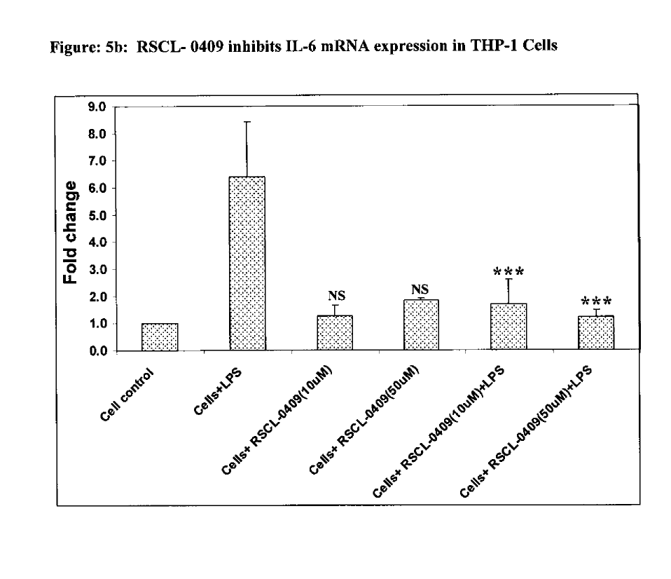

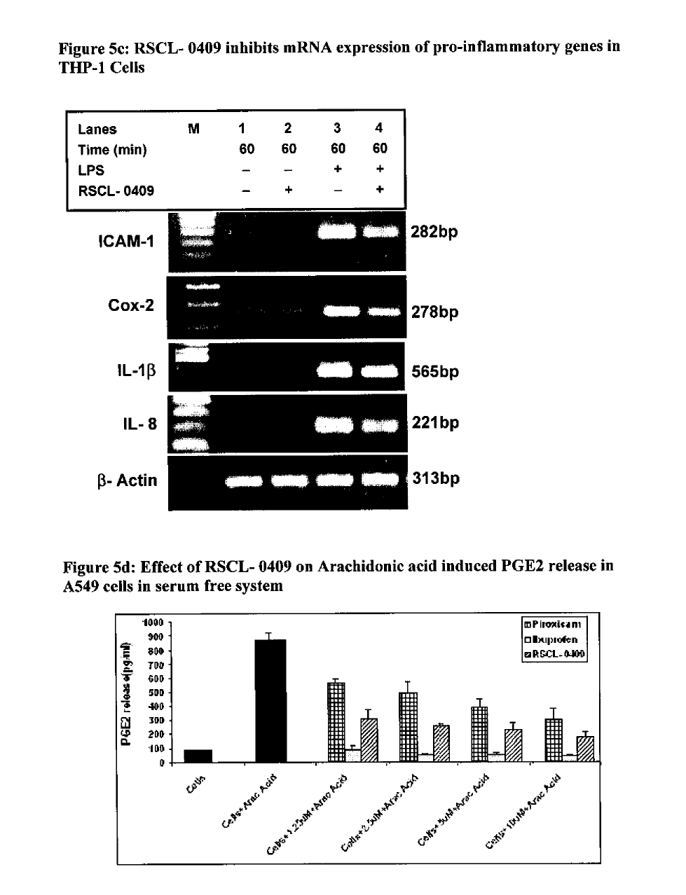

Quantization of mRNA was performed using the comparative threshold cycle method and analysed using standard software and expressed in fold change. The fold change of TNF-α (FIG. 5a) and IL-6 (FIG. 5b) mRNA in treated cells over control was obtained after correction for the amount of β-actin. We observed that RSCL-0409 down regulated TNF-α to almost control levels. The same was observed with IL-6 also in a concentration dependent manner. Cells treated with RSCL-0409 in the absence of LPS showed similar expression pattern as observed in untreated control cells. The expression levels of both TNF and IL-6 decreased following 1 hr pretreatment with RSCL-0409 in the presence of LPS. On the other hand, TNF-α and IL-6 mRNA expression increased rapidly after the stimulation with LPS. ***P<0.001 value is for LPS treated vs. RSCL-0409 treated followed by LPS treatment and NS is for Control Vs RSCL-0409.

Experiment 7: RSCL-0409 Inhibits mRNA Expression of Pro-Inflammatory Genes in THP-1 Cells.

The inhibitory effect of the present invention, RSCL-0409 on mRNA was checked on pro-inflammatory genes like intercellular adhesion molecule1 (ICAM-1), cyclooxygenase 2 (COX-2), IL-1β and IL-8. THP-1 cells (3×10⁶ cells) were seeded in a 6-well dish. The cells were treated with RSCL-0409 (50M) for 1 hr followed by incubation with or without 250 ng/ml of LPS. After two washes with ice-cold PBS, the cells were harvested and total cellular RNA was isolated using TRIZOL Reagent (Invitrogen) according to the manufacturer's instructions. cDNA synthesis was done using high capacity cDNA reverse transcription kit (ABI systems). Amplification of ICAM-1, COX-2, IL-1β and IL-8 genes from the cDNA was carried out using the respective gene specific primers.

RSCL-0409 inhibited mRNA expression levels of the tested genes (FIG. 5c), indicating that its mechanism of action is NF-κB mediated and highlights it's potential as a good anti-inflammatory agent. Cells treated with RSCL-0409 did not alter the gene expressions in any of the genes at mRNA level by itself in the absence of LPS treatment.

### Experiment 8: Effect of RSCL-0409 on Arachadonic Acid (AA) Induced PGE2 Release in A549 Cells.

COX-2 is the key enzyme regulating the production of prostaglandins, which act as central mediators of inflammation. Our earlier in-vitro data clearly demonstrated that the present invention inhibits expression of COX-2. It is well documented COX-2 pathway inhibitors were regarded as promising nonsteroidal anti-inflammatory drugs (NSAIDs). So we decided to test the ability of RSCL-0409 to block the COX-2 pathway to substantiate our earlier mRNA observation. For that purpose, we chose A549 cells, a human lung cancer cell line where COX-2 is activated by AA in serum-free stimulation established by Yao et al for 12 hr. (Yao J C, Duan W G, Yun Y, Liu de Q, Yan M, Jiang Z Z, Zhang L Y. Screening method for nonsteroidal antiinflammatory drugs based on the cyclooxygenase 2 pathway activated by serum-free stimulation in A549 cells. Yakugaku Zasshi. 2007; 127: 527-32.) In order to determine the efficiency of our molecule, we have incorporated two known COX-2 NSAIDs-Piroxicam and Ibuprofen for comparison purposes. A549 cells (50×10⁴ cells/well) in serum free medium were pretreated with different concentrations (10 μM, 5 μM, 2.5 μM, and 1.25 μM) of the NSAIDs and RSCL-0409 for 30 min. Then the cells were incubated with AA (10M) for another 30 min. Prostaglandin E2 (PGE2), a metabolite of AA through the Cox pathway, was assayed in an enzyme immunoassay (EIA) kit from R&D systems.

The results (FIG. 5d) indicate that RSCL-0409 shows a concentration dependent inhibition of PGE2 release. RSCL-0409 is effective even at 1.25 μM. In comparison with the NSAIDs, its inhibition of PGE2 is better than Piroxicam (at all the concentrations used), further enhancing it's potential as potent anti-inflammatory molecule. Further, reports have shown that lack of TLR4 reduces atherosclerosis in APO-E (−/−) mice and is associated to reduced COX-2 in these lesions. (Michelsen K S, Wong M H, Shah P K, Zhang W, Yano J, Doherty T M, Akira S, Rajavashisth T B, Arditi M. Lack of Toll-like receptor 4 or myeloid differentiation factor 88 reduces atherosclerosis and alters plaque phenotype in mice deficient in apolipoprotein E. Proc Natl Acad Sci USA. 2004; 101: 10679-10684.) Since the current molecule is a TLR4 antagonist, it could potentially be a candidate in treating atherosclerosis.

### Experiment 9: Effect of RSCL-0409 on LPS Induced Nitric Oxide (NO) Release in RAW 264.7 Cells

RAW264.7 cells provide an excellent model for evaluations of potential inhibitors on the pathway leading the induction of iNOS and NO production. Nitric oxide production was determined in RAW264.7 cells from the National Center of Cell Science (NCCS, Pune) cultured in color-free DMEM with standard supplements by measuring the amount of nitrite from cell culture supernatant. RAW264.7 cells (5×10⁴ per well) were stimulated for 24 hr with or without LPS (250 ng/ml) in the absence of presence of the RSCL-0409. Nitrite was then measured using the Griess reaction. 100 μl of cell culture supernatant was reacted with 100 μl of Griess reagent followed by spectrophotometric measurement at 550 nm. Nitrite concentrations in the supernatants were determined by comparison with a sodium nitrite standard curve. Alongside RSCL-0409 we also used known strong antioxidants (Vit C, Ascorbic acid, Catechin and Trolox) which inhibit NO to comparative antioxidant efficiency.

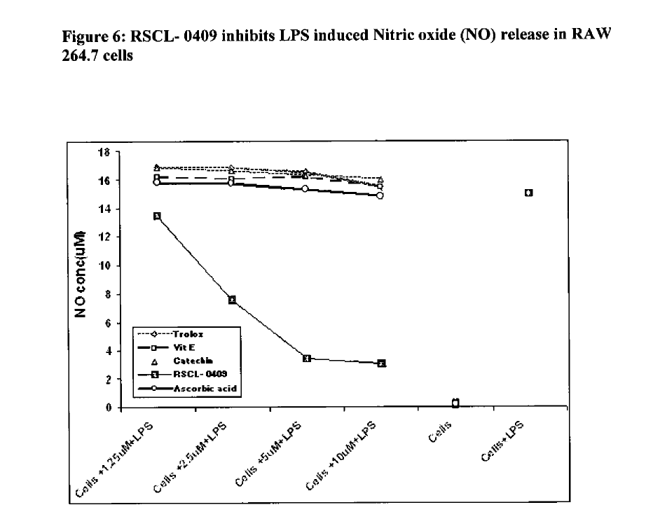

Results (FIG. 6) show that NO secretion induced by LPS stimulation was inhibited by RSCL-0409 in a concentration dependent manner. Further, the data clearly indicates that the anti-oxidant activity of RSCL-0409 was far more potent that those exhibited by the reference antioxidants used in our assay system.

### Experiment 10: Effect of RSCL-0409 on Activation of NEMO, Degradation of IκB-α and Activation of NF-κB.

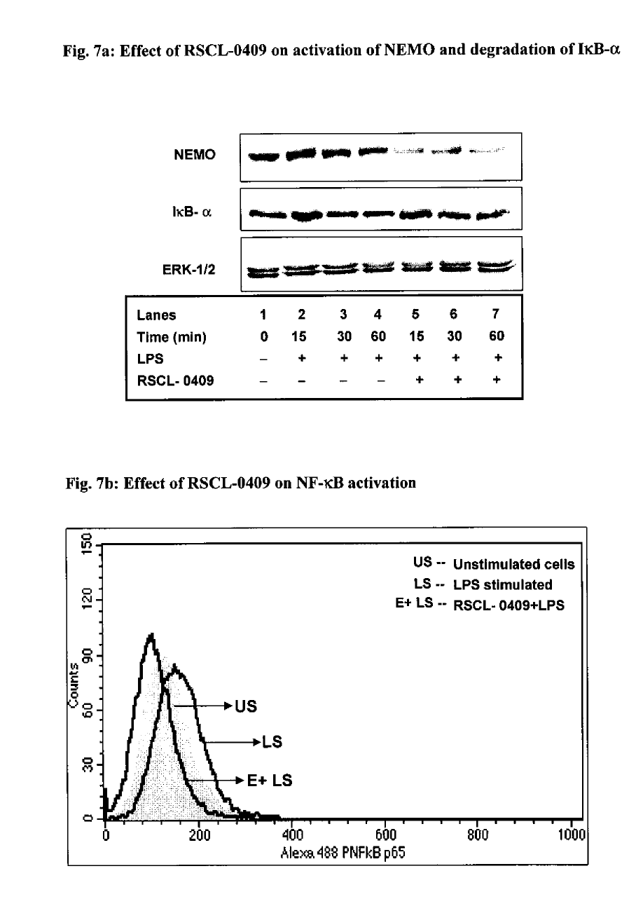

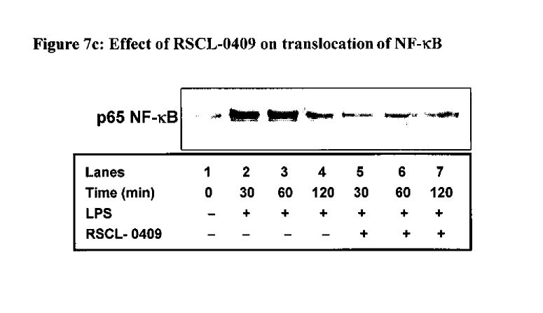

NF-κB/IκB complexes are present in the cytoplasm under unstimulated conditions. Following stimulation with LPS, we see phosphorylation and subsequent degradation of IκB allowing the free NF-κB to translocate into the nucleus to activate genes with NF-κB binding regions. Therefore, the effect of RSCL-0409 on blocking of NF-κB nuclear translocation was checked. Serum-starved THP-1 cells were stimulated with LPS (250 ng/ml) for the indicated time (FIG. 7a) in the presence and absence of RSCL-0409 (50M). RSCL-0409 treatment was 1 hr prior to LPS treatment. Total protein was isolated from the treated cells, and an equal amount of protein from each sample was used for immunoblots to determine protein levels of NEMO and IκB-α. The blot was stripped and reprobed with an anti-ERK-1/2 antibody to ensure equal loading. RSCL-0409 blocked signaling to NEMO, possibly blocking phosphorylation of IKK. This leads to inhibition of phosphorylation and further blocking p65 dissociation from IκB-α. Thus resulting in accumulation of IκB-α and inhibition of subsequent down stream signaling pathways.

Further, for the detection of intracellular location of phospho NF-κB p65 subunits, 1 hr LPS-stimulated cells with and without pretreatment of RSCL-0409 (50 μM—FIG. 7b) were fixed with 4% paraformaldehyde in PBS for 30 min and washed with Fluorescence-activated cell sorting (FACS) buffer [2% Fetal calm serum (FCS) in 1× Phosphate buffered saline (PBS)]. Permeabilisation is done using 90% methanol. These cells were then treated with phospho p65 monoclonal antibody tagged with Alexa Fluor 488 (Cell Signaling Technology, Inc, Ma, USA) for 1 hr at 37° C., followed by washing FACS buffer. Cells were then resuspended in PBS and acquired in BD FACS Calibur. Results substantiate the observation regarding RSCL-0409 inhibitory role in NF-κB signaling.

The nuclear fractions were obtained from LPS stimulated THP-1 cells at the indicated times (FIG. 7c). Nuclear proteins electrophoresed were processed for immunoblots using a NF-κB specific antibody. Immunoblot data (FIG. 7c) clearly shows that RSCL-0409 blocked translocation of NF-κB into the nucleus.

### Experiment 11: Effect of RSCL-0409 on NF-κB Activation in SEAP Reporter Assay

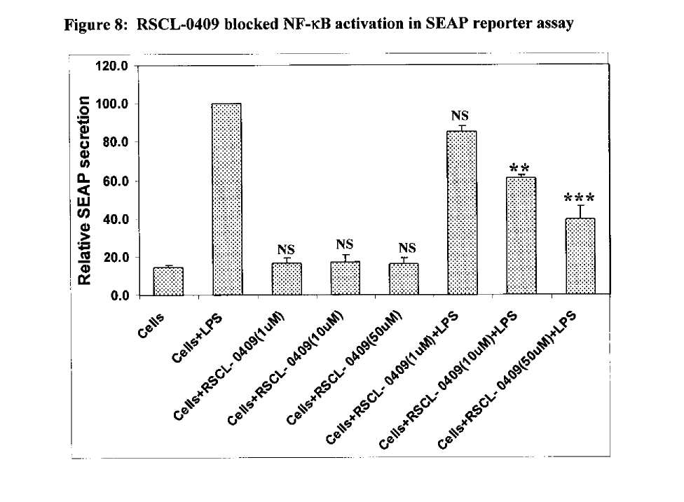

THP-1 CD14 Blue cells (Invivogen, San Diego, Calif., USA) transfected with a SEAP reporter construct in which the reporter expression was regulated by the NF-κB promoter were stimulated with LPS (250 ng/ml) with or without RSCL-0409 for 24 hr. SEAP gene on stimulation with a TLR4 ligand, LPS released large amounts of SEAP into culture medium which was blocked in a dose dependent manner by pretreatment of cells with RSCL-0409. The reporter activity was determined using Quanti Blue kit (Invivogen). The data (FIG. 8) is plotted as the relative change of reporter activity. NS; not significant, ** P value <0.01, *** P value <0.001 are statistics for Cells Vs RSCL-0409 treated and LPS treated vs. RSCL-0409 followed by LPS treatment respectively. These observations further confirm that the RSCL-0409 inhibits LPS induced TLR mediated activation of NF-κB transcription factor.

### Experiment 12: Effect of RSCL-0409 on MyD88 Dependent TLR Signaling

Serum-starved THP-1 cells were stimulated with LPS (250 ng/ml) for 1 hr in the presence and absence of RSCL-0409. Total RNA was isolated from treated cells post LPS exposure. The cDNA was used for PCR against specific primers for the TLR related genes and β-actin was used as internal control.

Narrowing down the likely TLRs involved, we have studied the intracellular signaling accessory/adaptor molecules involved in TLR signaling. Literature describes that LPS signaling through TLRs involves of four adaptor molecules myeloid differentiation primary response protein 88 (MyD88), Toll receptor IL-1R domain-containing adapter protein (TIRAP), TIR domain-containing adapter inducing IFNβ (TRIF), and Trif-related adapter molecule (TRAM). (Dunne A, O'Neill L A. Adaptor usage and Toll-like receptor signaling specificity. FEBS Lett. 2005; 579: 3330-3335.) Further, two signaling pathways; MyD88-dependent and MyD88-independent pathways have been elucidated downstream of TLR2 and 4, with MyD88 dependent pathway has been shown to be the most predominant signaling path demonstrated for TLR2 and TLR4 receptors. Applicants show that the present invention exerts its inhibitory effect in a MyD88 dependent manner.

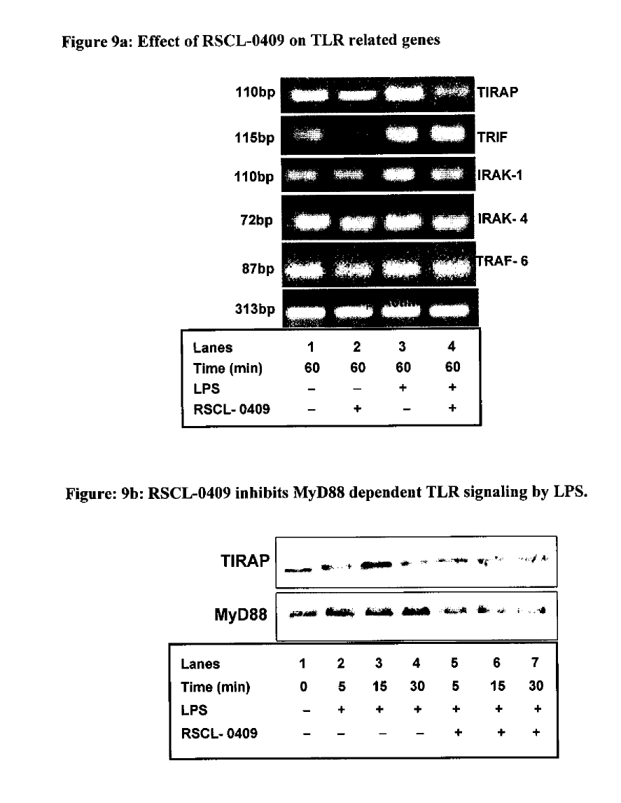

All these TLR related genes are upregulated upon stimulation with LPS (FIG. 9a lane 3); while pretreatment with RSCL-0409 inhibits the mRNA expression levels of TIRAP, IL-1R-associated kinase1 (IRAK1) and IRAK4. However the expression level of tumor necrosis factor receptor-associated factor 6 (TRAF6) was not down regulated significantly (FIG. 9a, lane 4). To rule out the possibility of a TRIF dependent pathway, a MyD88 independent path, we have checked its expression levels also in the treated cells. TRIF mRNA levels remained unaffected with pre-treatment of RSCL-0409. However, treatment of cells with RSCL-0409 did not show any effect in any of the genes at both mRNA level. These data suggest that RSCL-0409 inhibits MyD88 dependent signaling of TLR2/TLR4 by LPS.

Further confirmation was obtained at the protein level. Serum-starved THP-1 cells were stimulated with LPS (250 ng/ml) for the indicated time in the presence and absence of RSCL-0409. RSCL-0409 treatment was 1 hr prior to LPS treatment. Immunoblotting of total protein was carried out as before to determine protein levels of TIRAP and MyD88. Down regulation of TIRAP and also MyD88 at protein levels (similar to mRNA level) following LPS stimulation was observed in RSCL-0409 pretreated cells suggesting that RSCL-0409 inhibits MyD88 dependent TLR signaling by LPS (FIG. 9b).

### Example 3

### In Vivo Testing of RSCL-0409

### Experiment 1: Effect of RSCL-0409 on LPS Induced TNF-αc Release in Balb/c Mice

The present invention has studied the ability of RSCL-0409 to exert protection against inflammatory agents in a mice (Balb/c) model. Balb/c (5-6 weeks) mice were injected with LPS (225 μg) intraperitoneally with and without pretreatment of three concentrations of RSCL-0409 (10 mg/kg, 20 mg/kg and 40 mg/kg). The compound was injected intraperitoneally 30 min before LPS treatment. The mice were monitored for 1 hr post LPS treatment. RSCL-0409 injected alone served as negative control. Blood collection was done retro-orbitally under anesthesia, 1 hr post LPS injection. Serum collected after cells were allowed to settle was analysed for TNF-α secretion.

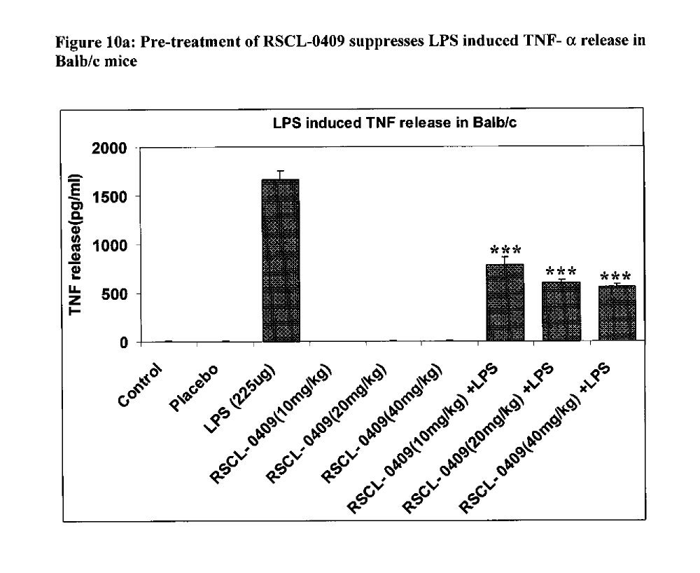

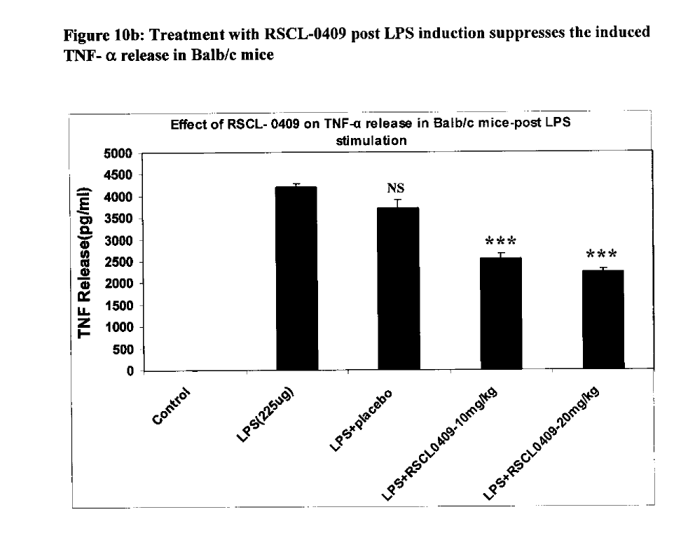

In untreated mice, LPS injection led to the secretion of large amounts on TNF-α in the serum. However, pretreated mice significantly reduced TNF production (˜53%, 64% and 67% respectively, FIG. 10a) reconfirming our in-vitro data. *** P value <0.001 represents LPS treated vs. RSCL-0409 treated. Control represents untreated animals. In this context, the present invention shows potential clinical application to effectively attenuate LPS mediated TNF useful in preventing onset of sepsis.

Further authentication of the current invention being a potent molecule exhibiting promising anti-inflammatory activity is shown in FIG. 10b. Here we have stimulated the inflammation process in mice with an injection of LPS (225 μg) intraperitoneally and 30 min post LPS, we have challenged the ability of RSCL-0409 (10 mg/kg, 20 mg/kg) to inhibit the TNF-α secretion. We see at both doses there is inhibition of TNF secretion (˜39% and 47% respectively). *** P value <0.001 represents LPS treated vs. RSCL-0409 treated, NS represents non significant.

The results indicate that the compound of the present invention exhibit promising In-vitro and In-vivo activity with both results being corroborative and clearly showing our molecule to be a potent TLR antagonist and anti-inflammatory molecule.

All publications and patent applications cited in this specification are herein incorporated by reference as if each individual publication or patent application are specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

We claim:

1. A compound of formula I: 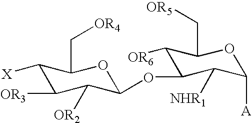
wherein
at least one or all of R1, R2, R3 or R4 are selected from: 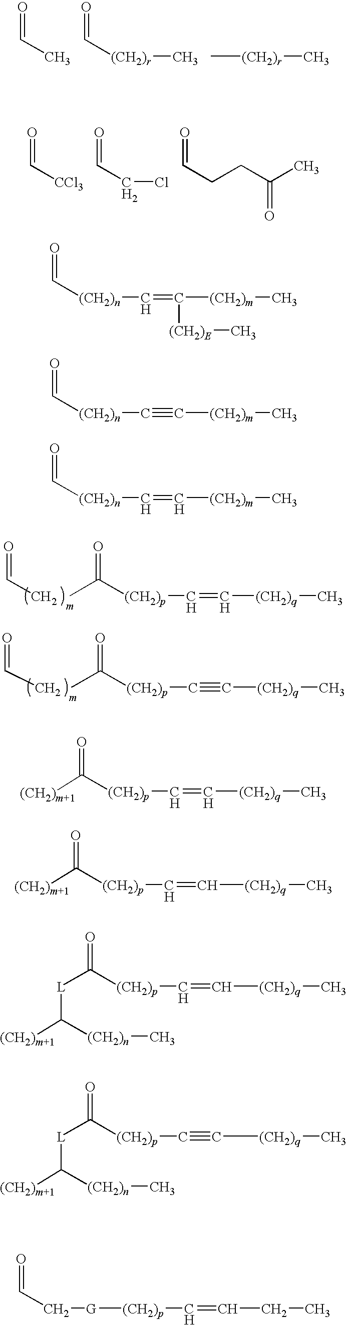 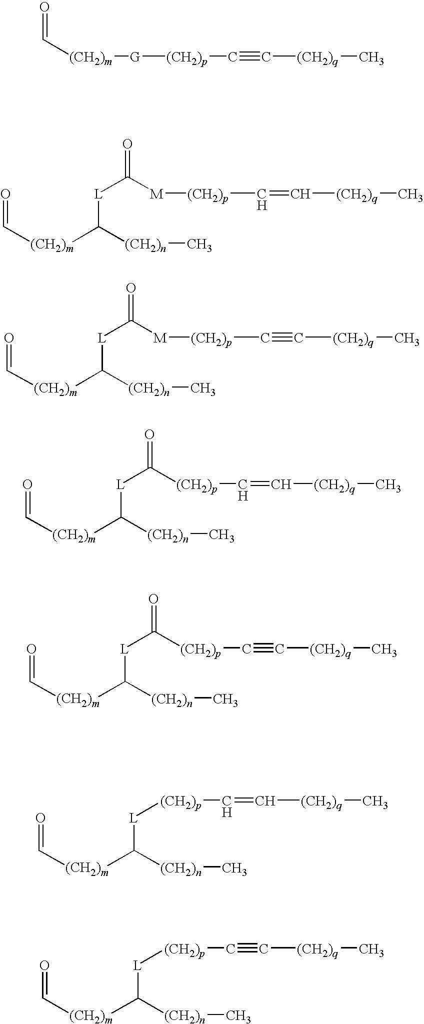
each L is O, N or C;
each M is O or N;
each E, independently, is an integer between 0 and 14 inclusive;
each G, independently, is N, O, S, SO, or SO₂;
each m, independently, is an integer between 0 and 14 inclusive;
each n, independently, is an integer between 0 and 14 inclusive;
each p, independently, is an integer between 0 and 10 inclusive;
each r, independently is an integer between 0 and 20 inclusive;
each q, independently, is an integer between 0 and 10 inclusive;
each of the remaining R1, R2, R3, and R4, independently, is: 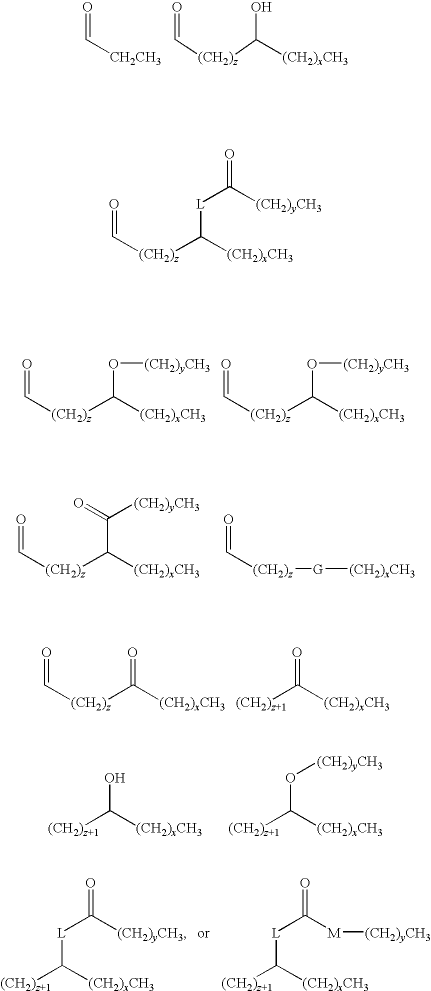
wherein
each L is O, N or C;
each M is O or N;
each x, independently, is an integer between 0 and 14 inclusive;
each y, independently, is an integer between 0 and 14 inclusive;
each z, independently, is an integer between 0 and 10 inclusive; and
each G, independently, is N, O, S, SO, SO₂;
each A and X, independently, is H, OH, OCH₃, C₆H₅OCH₃, 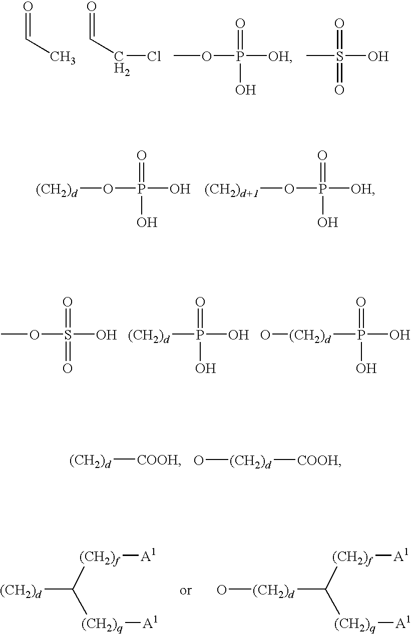
wherein
each d, independently, is an integer between 0 and 5 inclusive;
each f, independently, is an integer between 0 and 5 inclusive;
each g, independently, is an integer between 0 and 5 inclusive; and
each A¹, independently, is 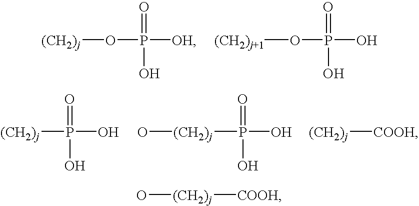
wherein
each j, independently, is an integer between 0 and 14 inclusive;
X is OH, Cl, O(CH₂)ₜCH₃, O(CH₂)ₜOH, O(CH₂)ₜO(CH₂)ᵥCH₃ 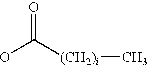
wherein
i, independently, is an integer between 0 and 20 inclusive;
each t and v, independently, is an integer between 0 and 14 inclusive;
R₅ and R₆ is any of R₁-R₄, H, benzylidene, or acetonide, and polymorphs, isomers, prodrugs, pharmaceutically acceptable salts, amides and esters thereof,
wherein the compound is an antagonist of a toll-like receptor (TLR).

2. The compound of claim 1 having the structure:

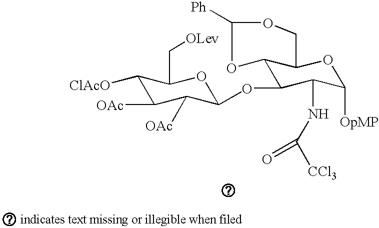

3. p-Methoxy phenyl-O-(2,3-di-O-acetyl-4-O-chloroacetyl-6-O-levulinoyl-β-D-glucopyranosyl)-(1-3)-2-deoxy-2-trichloroacetamido-4,6-O-benzylidene-α-D-glucopyranoside (RSCL-0409) and polymorphs, isomers, prodrugs, pharmaceutically acceptable salts, amides and esters thereof.

4. A pharmaceutical composition for use in preparing a medicament, the composition comprising an effective amount of a compound according to any of claims 1-3, and a pharmaceutically acceptable carrier, adjuvant or diluent.

5. The pharmaceutical composition of claim 3, for use in preparing a medicament for prevention or treatment of an immunological disease or condition in a mammal.

6. The pharmaceutical composition of claim 3, for use in preparing a medicament for prevention or treatment of an infectious or inflammatory disease in a mammal.

7. The pharmaceutical composition of claims 4 or 5, wherein the mammal is human.

8. The pharmaceutical composition of claim 4, comprising an effective amount of the compound sufficient for inhibiting a toll like receptor (TLR).

9. The pharmaceutical composition of claim 7, comprising an effective amount of the compound sufficient for inhibiting TLR4.

10. The pharmaceutical composition of claim 4, comprising an effective amount of the compound sufficient for modulating an immune response.

11. The pharmaceutical composition of claim 9, comprising an effective amount of the compound sufficient for prevention or treatment of a lipopolysaccharide (LPS)-mediated disease selected from the group consisting of inflammatory bowel disease (IBD), sepsis, periodontal disease, mucositis, acne, cardiovascular disease, chronic obstructive pulmonary disease, arthritis, cystic fibrosis, bacterial-induced infections, viral-induced infections, mycoplasma-associated diseases, post herpetic neuralgia, ischemia/reperfusion injury, asthma, stroke, brain injury, necrotizing enterocolitis, bed sores, leprosy, atopic dermatitis, psoriasis, trauma, neurodegenerative disease, amphotericin B-induced fever and nephritis, coronary artery bypass grafting, and atherosclerosis.

12. The pharmaceutical composition of claim 9, comprising an effective amount of the compound sufficient for prevention or treatment of an autoimmune disorder selected from the group consisting of: systemic lupus erythematosus, rheumatoid arthritis, inflammatory bowel disease, Sjogren's syndrome, polymyositis, vasculitis, Wegener's granulomatosis, sarcoidosis, ankylosing spondylitis, Reiter's syndrome, psoriatic arthritis, and Behget's syndrome.

13. The pharmaceutical composition of claim 9, comprising an effective amount of the compound sufficient for prevention or treatment of inflammation, wounds, autoimmunity, allergy, asthma, graft rejection, graft versus host disease, infection, sepsis, cancer, or immunodeficiency.

14. The pharmaceutical composition of claim 4, comprising an effective amount of the compound sufficient for modulating immune system activity mediated by Toll-like receptors (TLRs).

15. The pharmaceutical composition of claim 4, comprising an effective amount of the compound sufficient for inhibition of TLR signaling in response to TLR ligand or TLR signaling agonist.

16. The pharmaceutical composition of claims 4, 13 or 14, comprising an effective amount of the compound sufficient for inhibition of TLR4 signaling under physiological conditions.

17. An unit dose of the pharmaceutical composition of claim 3, comprising 0.01 to 100 mg of the compound per kg of adult body weight.

18. An article of manufacture comprising the pharmaceutical composition of claim 3 provided in a form suitable for administration to a patient in need thereof.

19. A formulation comprising (a) the pharmaceutical composition of claim 4 and (b) a second agent affecting non-antigen presenting cells bearing TLRs, in amounts sufficient to exhibit a synergistic effect on TLR-mediated immunostimulation.

20. The pharmaceutical composition of claim 5, comprising an effective amount of the compound sufficient for inhibition of cyclooxygenase 2 (COX-2) pathway.

21. The pharmaceutical composition of claim 5, comprising an effective amount of the compound sufficient for inhibition of nitric oxide (NO) release during inflammation caused by immunostimulatory molecules.

22. The pharmaceutical composition of claim 5, comprising an effective amount of the compound sufficient for inhibition of nitric oxide (NO) release during inflammation caused by lipopolysaccharides (LPS).

23. The pharmaceutical composition of claims 5 or 22, comprising an effective amount of the compound sufficient for prevention or treatment of an inflammatory disease selected from the group consisting of rheumatoid arthritis, bacterial infections, side effects of bacterial infections, sepsis, atherosclerosis and inflammatory bowel disease (IBD).

24. The pharmaceutical composition of claims 5 or 22, comprising an effective amount of the compound sufficient for inhibition of expression of pro-inflammatory genes selected from intercellular adhesion molecule1 (ICAM-1), cyclooxygenase 2 (COX-2), IL-1β and IL-8.

25. The pharmaceutical composition of claims 5 or 22, comprising an effective amount of the compound sufficient for inhibition of LPS induced TNF-alpha release.

26. The pharmaceutical composition of claims 5 or 22, comprising an effective amount of the compound sufficient for inhibition of degradation of IκB-α or activation of NF-κB.

27. A method for modulating immune system activity mediated by Toll-like receptors (TLRs) comprising contacting a cell expressing a TLR with an effective amount of p-Methoxy phenyl-O-(2,3-di-O-acetyl-4-O-chloroacetyl-6-O-levulinoyl-β-D-glucopyranosyl)-(1-3)-2-deoxy-2-trichloroacetamido-4,6-O-benzylidene-α-D-glucopyranoside (RSCL-0409) having the structure 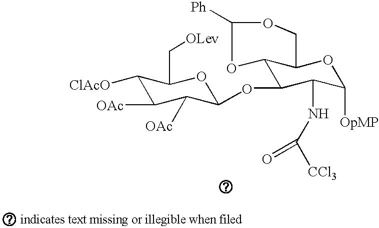
and polymorphs, isomers, prodrugs, pharmaceutically acceptable salts, amides and esters thereof.

28. A method for modulating inflammatory activity mediated by Toll-like receptors (TLRs) comprising contacting a cell expressing a TLR with an effective amount of the compound p-Methoxy phenyl-O-(2,3-di-O-acetyl-4-O-chloroacetyl-6-O-levulinoyl-β-D-glucopyranosyl)-(1-3)-2-deoxy-2-trichloroacetamido-4,6-O-benzylidene-α-D-glucopyranoside (RSCL-0409) having the structure: 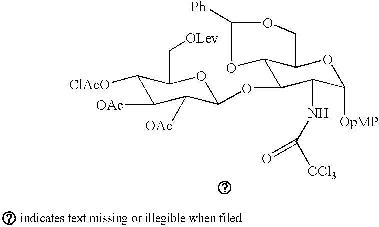
and polymorphs, isomers, prodrugs, pharmaceutically acceptable salts, amides and esters thereof.

29. The method of claim 28, comprising administering to an individual in need thereof, a therapeutically effective amount of the compound sufficient for prevention or treatment of an inflammatory disease selected from the group consisting of rheumatoid arthritis, bacterial infections, side effects of bacterial infections, sepsis, atherosclerosis and inflammatory bowel disease (IBD).

30. The method of claim 28, comprising administering to an individual in need thereof, a therapeutically effective amount of the compound sufficient for prevention or treatment of a non specific inflammatory reaction.

31. The method of claim 28, comprising administering to an individual in need thereof, a therapeutically effective amount of the compound sufficient for inhibition of expression of pro-inflammatory genes selected from intercellular adhesion molecule1 (ICAM-1), cyclooxygenase 2 (COX-2), IL-1 and IL-8.

32. The method of claim 28, comprising administering to an individual in need thereof a therapeutically effective amount of the compound sufficient for inhibition of LPS induced TNF-alpha release.

33. The method of claim 28, comprising administering to an individual in need thereof, a therapeutically effective amount of the compound sufficient for inhibition of degradation of IκB-α or activation of NF-κB.

34. The method of claim 28, comprising administering to an individual in need thereof, a therapeutically effective amount of the compound sufficient for treating or preventing a clinical condition or disease caused by a microbial pathogen, by administering a therapeutically effective amount of the compound to an individual in need thereof.

35. The method of claim 26, comprising administering to an individual in need thereof, a therapeutically effective amount of the compound sufficient for inhibiting a toll like receptor (TLR).

36. The method of claim 26, comprising administering to an individual in need thereof, a therapeutically effective amount of the compound sufficient for inhibiting TLR4.

37. The method of claim 26, comprising administering to an individual in need thereof, a therapeutically effective amount of the compound sufficient for modulating an immune response.

38. The method of claim 26, comprising administering to an individual in need thereof, a therapeutically effective amount of the compound sufficient for prevention or treatment of a lipopolysaccharide (LPS)-mediated disease selected from the group consisting of inflammatory bowel disease (IBD), sepsis, periodontal disease, mucositis, acne, cardiovascular disease, chronic obstructive pulmonary disease, arthritis, cystic fibrosis, bacterial-induced infections, viral-induced infections, mycoplasma-associated diseases, post herpetic neuralgia, ischemia/reperfusion injury, asthma, stroke, brain injury, necrotizing enterocolitis, bed sores, leprosy, atopic dermatitis, psoriasis, trauma, neurodegenerative disease, amphotericin B-induced fever and nephritis, coronary artery bypass grafting, and atherosclerosis.

39. The method of claim 26, comprising administering to an individual in need thereof, a therapeutically effective amount of the compound sufficient for prevention or treatment of an autoimmune disorder selected from the group consisting of: systemic lupus erythematosus, rheumatoid arthritis, inflammatory bowel disease, Sjogren's syndrome, polymyositis, vasculitis, Wegener's granulomatosis, sarcoidosis, ankylosing spondylitis, Reiter's syndrome, psoriatic arthritis, and Behget's syndrome.

40. The method of claim 26, comprising administering to an individual in need thereof, a therapeutically effective amount of the compound sufficient for prevention or treatment of inflammation, wounds, autoimmunity, allergy, asthma, graft rejection, graft versus host disease, infection, sepsis, cancer, or immunodeficiency.

41. The method of claim 26, comprising administering to an individual in need thereof, a therapeutically effective amount of the compound sufficient for inhibition of TLR signaling in response to TLR ligand or TLR signaling agonist.

42. The method of claim 26, comprising administering to an individual in need thereof, a therapeutically effective amount of the compound sufficient for inhibition of TLR4 signaling under physiological conditions.

43. The method of claims 26 or 28, comprising administering the compound by a route selected from oral, intraperitoneal, intramuscular, intravenous, intra-articular, intralesional, subcutaneous, nasal, rectal, buccal, or a route sufficient to provide an amount sufficient to inhibit TLR activity

44. A method for screening agents that inhibit toll-like receptor activation, the method comprising:
contacting a cell expressing a TLR with a candidate agent in the presence of a TLR activator or agonist; and
determining the effect of the candidate agent on activation of TLR,
wherein the candidate agent is a compound according to any of claims 1-3.

45. The method of claim 43, further comprising comparing the activity of the candidate agent with an activity of a compound according to any of claims 1-3.

46. The compounds, compositions, and methods for the preparation and antagonist of a toll-like receptor (TLR) as claimed above exemplified herein substantially in the examples and figures.
